(19) United States
(12) Patent Application Publication
(10) Pub. No.: US 2024/0000577 A1
(43) Pub. Date: Jan. 4, 2024

(54) CONVERTIBLE SHOULDER ARTHROPLASTY SYSTEMS AND RELATED METHODS

(71) Applicant: Encore Medical, LP DBA DJO Surgical, Austin, TX (US)

(72) Inventors: Stuart L. Axelson, Succasunna, NJ (US); Anthony Joseph La Rosa, Wharton, NJ (US); Vasilievich Romanov, Jersey City, NJ (US); Mark A. Frankle, Tampa, FL (US); Joseph P. Iannotti, Delray Beach, FL (US); Jonathan Levy, Fort Lauderdale, FL (US); Gerald Williams, Villanova, PA (US); Joseph A. Abboud, Bryn Mawr, PA (US); Thomas Brad Edwards, Houston, TX (US)

(21) Appl. No.: 18/216,155

(22) Filed: Jun. 29, 2023

Related U.S. Application Data
(60) Provisional application No. 63/356,625, filed on Jun. 29, 2022.

(51) Int. Cl. A61F 2/40; A61F 2/30; A61B 17/86

(52) U.S. Cl. CPC A61F 2/4081; A61F 2/30749; A61B 17/86; A61F 2002/30649; A61F 2002/4085; A61F 2002/30841; A61F 2002/30892; A61F 2002/30827; A61F 2310/00023; A61F 2310/00029; A61F 2002/305; A61B 2017/564

(57) ABSTRACT

Convertible shoulder arthroplasty systems and configurations for components thereof are described. In some embodiments, an implant component is provided. The implant component includes one of a glenoid component comprising a concave outer surface, and a glenosphere component comprising a convex outer surface. The system may also include one of a first baseplate comprising a central bossed portion extending from an underside thereof and a central aperture disposed therethrough or a second baseplate comprising a central bossed portion extending from a top side thereof and a central aperture disposed there through. In some implementations, an anchor boss comprising a central aperture extending therethrough is provided. The system may include a central compression screw configured to be secured through the central aperture and, thereby, securely compress the first baseplate, the second baseplate or the anchor boss against the patient bone.

### FIELD OF THE DISCLOSURE

The disclosure relates generally to surgical implant systems. More particularly, the disclosure relates to universal and/or convertible glenoid and/or glenosphere shoulder arthroplasty systems and related methods.

### BACKGROUND

Shoulder arthroplasty is a common method of repair for a shoulder joint that has become dysfunctional due to disease or trauma. In a healthy shoulder joint, the humeral head is generally ball-shaped and articulates within a socket formed by the scapula, called the glenoid cavity. Conventional implant systems for total replacement of the shoulder joint (e.g., total shoulder arthroplasty (TSA)) generally replicate the natural anatomy of the shoulder and include a metal humeral component having a stem which fits within the humeral canal, and a head that articulates within the socket of a plastic glenoid component implanted within the glenoid of the scapula. The glenoid component can be a single piece component attached to the glenoid, or a two-piece component having a plastic glenoid component attached to a metal baseplate that is, in turn, attached to the glenoid.

In some cases, it is only necessary to replace a part of the shoulder joint, for example, by replacing the humeral head (e.g., a hemi shoulder arthroplasty (HAS)) with a prosthetic humeral head configured to articulate within the natural glenoid cavity of the scapula.

In addition, “reverse” type implant systems (e.g., total reverse shoulder arthroplasty (RSA)) reverse the conventional ball-and-socket configuration by using a concave recessed articulating component at the proximal end of the humeral component which articulates against a convex portion of a ball-shaped component. In some applications, reverse shoulder implant systems can provide increased range of motion for treatment of glenoid humeral arthritis associated with irreparable rotator cuff damage. RSA may also be indicated for some cases of advanced bone loss or damage.

Given the total, partial and reverse types of shoulder arthroplasty, a need exists for universal or convertible arthroplasty systems and related methods that allow a practitioner to perform, revise, or convert any of the types of shoulder arthroplasty utilizing a same set of convertible or universal components and/or tools.

### SUMMARY

According to some example embodiments, an implant component is provided. The implant component includes one of a glenoid component comprising a concave, arcuate top surface, and a glenosphere component comprising a convex outer surface. The implant component includes one of: a glenoid baseplate comprising a central bossed portion extending from an underside thereof and a central aperture disposed therethrough, a glenosphere baseplate comprising a central bossed portion extending from a top side thereof and a central aperture disposed there through, and an anchor boss comprising a central aperture extending therethrough. The implant component includes a central compression screw configured to be secured through the central aperture and, thereby, securely compress the glenoid baseplate, the glenosphere baseplate or the anchor boss against the patient bone.

According to some example embodiments, a convertible shoulder arthroplasty system is provided. The system includes a central anchor screw comprising threads configured to bite into patient bone and provide a stand-alone anchor therein. The system includes one of: a first glenoid component comprising a concave, arcuate top surface and an underside comprising a metal disk-like component disposed therein that provides a key locking interface mating the glenoid component and the central anchor screw, a glenoid baseplate comprising a substantially planar top surface, a central aperture disposed therethrough, and an underside comprising the metal disk-like component disposed therein that provides a key locking interface mating the glenoid baseplate and the central anchor screw; and a glenosphere baseplate comprising a central bossed portion extending from a top side thereof, the central aperture disposed therethrough, and an underside comprising the metal disk-like component disposed therein that provides a key locking interface mating the glenosphere baseplate and the central anchor screw.

According to some example embodiments, a method of utilizing a convertible shoulder arthroplasty system to perform shoulder arthroplasty is provided. The method includes drilling a central hole into a resected proximal end of a humerus of a patient, the central hole configured to receive a guide wire. The method includes preparing a surface of the resected proximal end of the humerus utilizing a reamer disposed over the guide wire. The method includes drilling out the central hole to accommodate at least a portion of a baseplate or an anchor boss and a central compression screw of the system. The method includes securing the central compression screw through a central aperture in the baseplate or in the anchor boss and into the central hole to, thereby, secure the baseplate or the anchor boss against the resected proximal end of the humerus. The method includes coupling to the baseplate or to the anchor boss, one of a glenoid component comprising a concave, arcuate top surface, and a glenosphere component comprising a convex outer surface.

According to some example embodiments, another method of utilizing a convertible shoulder arthroplasty system to perform shoulder arthroplasty is provided. The method includes drilling a central hole into a surface of a scapula of a patient, a proximal portion of the central hole having a larger radius than portions of the central hole distal of the proximal portion to accommodate a central anchor screw entirely therein. The method includes securing the central anchor screw entirely in the central hole. The method includes disposing a guide wire or a guide wire guide coupled to the guide wire into a head of the central anchor screw. The method includes preparing the surface of the scapula utilizing a reamer disposed over the guide wire. The method includes coupling to the head of the central anchor screw after the guide wire is removed, one of: a first glenoid component comprising a concave, arcuate top surface and an underside comprising a metal disk-like component disposed therein that provides a key locking interface mating the glenoid component and the central anchor screw, a glenoid baseplate comprising a substantially planar top surface, a central aperture disposed therethrough, and an underside comprising the metal disk-like component disposed therein that provides a key locking interface mating the glenoid baseplate and the central anchor screw, and a glenosphere baseplate comprising a central bossed portion extending from a top side thereof, the central aperture disposed therethrough, and an underside comprising the metal disk-like component disposed therein that provides a key locking interface mating the glenosphere baseplate and the central anchor screw.

According to some example embodiments, a method of manufacturing a convertible shoulder arthroplasty system is provided. Such a method may include, without limitation, providing, forming, fabricating, molding including but not limited to injecting molding or overmolding, extruding, stamping, deforming, casting, forging, milling, machining, printing including but not limited to 3-D printing any element and/or feature of any component, or the component itself to, thereby manufacture any component(s) described in this disclosure. Accordingly, manufacturing any component may comprise any one or more of these actions or steps, and contrarily, any one or more of these actions or steps may be considered manufacturing such a component and/or element thereof.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 is a side expanded view of a convertible modular system for shoulder arthroplasty, in accordance with some example embodiments;

FIG. 2 is an assembled perspective view from the bottom of the convertible modular system of FIG. 1;

FIG. 3 is a side expanded view of another convertible modular system for shoulder arthroplasty, in accordance with some example embodiments;

FIG. 4 is an assembled perspective view from the bottom of the convertible modular system of FIG. 3;

FIG. 5 is a side expanded view of yet another convertible modular system for shoulder arthroplasty, in accordance with some example embodiments;

FIG. 6 is an assembled perspective view from the bottom of the convertible modular system of FIG. 5;

FIG. 7 is a side expanded view of yet another convertible modular system for shoulder arthroplasty, including an expanded perspective view of a glenoid component thereof, in accordance with some example embodiments;

FIG. 8 is an assembled perspective view from the bottom of the convertible modular system of FIG. 7;

FIG. 9 is a side expanded view of yet another convertible modular system for shoulder arthroplasty, in accordance with some example embodiments;

FIG. 10A is an expanded perspective view from the top of the convertible modular system of FIG. 9;

FIG. 10B is an expanded perspective view from the bottom of the convertible modular system of FIG. 9;

FIG. 11 illustrates a variety of perspective views of a bottom side of the glenoid component and metal disk-like component of FIGS. 9-10B;

FIG. 12 is an expanded perspective view from the top of yet another convertible modular system for shoulder arthroplasty, in accordance with some example embodiments;

FIG. 13 is an expanded perspective view from the top of a multi-piece glenoid component of the convertible modular system for shoulder arthroplasty of FIG. 14;

FIG. 14 is an expanded perspective view from the top of yet another convertible modular system for shoulder arthroplasty, in accordance with some example embodiments;

FIG. 15 is an expanded perspective view from the bottom of yet another convertible modular system for shoulder arthroplasty, in accordance with some example embodiments;

FIG. 16A is a cutaway side view of the convertible modular system of FIG. 15;

FIG. 16B is a top view of the convertible modular system of FIG. 16A;

FIG. 16C is a cutaway magnified side view of a portion of the convertible modular system as viewed along the cutline A-A in FIG. 16B;

FIG. 16D is a partially transparent perspective view from the top of a portion of the convertible modular system of FIG. 16A;

FIG. 16E is a perspective view from the top of convertible modular system of FIG. 16A;

FIG. 17 is an expanded perspective view of yet another convertible modular system for shoulder arthroplasty, in accordance with some example embodiments;

FIG. 18A is an expanded perspective view from the bottom of yet another convertible modular system for shoulder arthroplasty, in accordance with some example embodiments;

FIG. 18B is an expanded perspective view from the bottom of yet another convertible modular system for shoulder arthroplasty, in accordance with some example embodiments;

FIG. 19A is an expanded perspective view from the bottom of yet another convertible modular system for shoulder arthroplasty, in accordance with some example embodiments;

FIG. 19B is an expanded perspective view from the bottom of yet another convertible modular system for shoulder arthroplasty, in accordance with some example embodiments;

FIG. 20 is a partially expanded perspective view from the bottom of yet another convertible modular system for shoulder arthroplasty, in accordance with some example embodiments;

FIG. 21 is an expanded side view from the bottom of yet another convertible modular system for shoulder arthroplasty, in accordance with some example embodiments;

FIG. 22A is an expanded perspective view from the bottom of several components of the convertible modular system of FIG. 21;

FIG. 22B is an assembled perspective view from the bottom of the convertible modular system of FIG. 21;

FIG. 23A is an expanded perspective view from the bottom of yet another convertible modular system for shoulder arthroplasty, in accordance with some example embodiments;

FIG. 23B is a cutaway side view of several components of the convertible modular system of FIG. 23A;

FIG. 24 is a perspective side view of the central compression screw of the convertible modular system of FIG. 21 and a screw snap ring;

FIG. 25 is an expanded perspective side view of a convertible modular system comprising the system of FIG. 21 and the screw snap ring of FIG. 24;

FIG. 26A is an expanded perspective view from the bottom of the convertible system of FIG. 25;

FIG. 26B is an expanded perspective view from the top of the convertible system of FIG. 25;

FIG. 27 illustrates an example combination of a baseplate and central compression screw for use in a convertible modular system for shoulder arthroplasty, in accordance with some example embodiments;

FIG. 28 illustrates another example combination of a baseplate and central compression screw for use in a convertible modular system for shoulder arthroplasty, in accordance with some example embodiments;

FIG. 29 illustrates yet another example combination of a baseplate and central compression screw for use in a convertible modular system for shoulder arthroplasty, in accordance with some example embodiments;

FIG. 30 illustrates yet another example combination of a baseplate and central compression screw for use in a convertible modular system for shoulder arthroplasty, in accordance with some example embodiments;

FIG. 31 illustrates a side view and perspective view from the top of an example baseplate for use in a convertible modular system for shoulder arthroplasty, in accordance with some example embodiments;

FIG. 32 illustrates a side view and perspective view from the top of another example baseplate for use in a convertible modular system for shoulder arthroplasty, in accordance with some example embodiments;

FIG. 33 illustrates a side view and perspective view from the top of yet another example baseplate for use in a convertible modular system for shoulder arthroplasty, in accordance with some example embodiments;

FIG. 34 illustrates a side view and perspective view from the top of yet another example baseplate for use in a convertible modular system for shoulder arthroplasty, in accordance with some example embodiments;

FIG. 35A is a side view of several components of a convertible modular system for shoulder arthroplasty that includes a baseplate wedge, in accordance with some example embodiments;

FIG. 35B is a cutaway side view of the system components of FIG. 35A;

FIG. 35C is a top view of the system components of FIG. 35A;

FIG. 35D is a top perspective view of the system components of FIG. 35A;

FIG. 35E is another top perspective view of the system components of FIG.

FIG. 35F is a bottom perspective view of the system components of FIG.

FIG. 35G is another bottom perspective view of the system components of FIG. 35A;

FIG. 36 illustrates a side view of several features of example glenosphere components for utilization in any compatible convertible modular system for shoulder arthroplasty described herein;

FIG. 37 is a side view of a convertible modular system for shoulder arthroplasty, in accordance with some example embodiments;

FIG. 38 is a cutaway side view of the convertible modular system of FIG. 37;

FIG. 39A illustrates side and bottom perspective views of a convertible modular system for shoulder arthroplasty, in accordance with some example embodiments;

FIG. 39B illustrates side and bottom perspective views of another convertible modular system for shoulder arthroplasty, in accordance with some example embodiments;

FIG. 40A illustrates side and bottom perspective views of yet another convertible modular system for shoulder arthroplasty, in accordance with some example embodiments;

FIG. 40B illustrates side and bottom perspective views of yet another convertible modular system for shoulder arthroplasty, in accordance with some example embodiments;

FIG. 41A is an expanded bottom perspective view of a convertible modular system for shoulder arthroplasty, in accordance with some example embodiments;

FIG. 41B is an expanded top perspective view of the convertible modular system of FIG. 41A;

FIG. 42 is a cutaway side view of portions of a convertible modular system for shoulder arthroplasty utilizing a central compression screw having a non-threaded head, in accordance with some example embodiments;

FIG. 43 is a cutaway side view of portions of another convertible modular system for shoulder arthroplasty utilizing a central compression screw having a threaded head, in accordance with some example embodiments;

FIG. 44 is an expanded top perspective view of a convertible modular system for shoulder arthroplasty employing a set screw to prevent central compression screw backout, in accordance with some example embodiments;

FIG. 45 is an expanded side view of a convertible modular system for shoulder arthroplasty employing a threaded glenosphere adapter to prevent central compression screw backout, in accordance with some example embodiments;

FIG. 46 is an expanded bottom perspective view of a convertible modular system for shoulder arthroplasty, in accordance with some example embodiments;

FIG. 47 is an expanded side view of another convertible modular system for shoulder arthroplasty, in accordance with some example embodiments;

FIG. 48 illustrates a partial assembled top perspective view and a partial bottom perspective view of several components of a convertible modular system for shoulder arthroplasty, in accordance with some example embodiments;

FIG. 49 illustrates a surgical technique overview for preparing a surface of patient humeral bone for using a convertible modular system for shoulder arthroplasty, in accordance with some example embodiments;

FIG. 50A is a top perspective view of a portion of patient scapular bone being prepared for a convertible modular system for shoulder arthroplasty, in accordance with some example embodiments;

FIG. 50B is a cutaway side view of the portion of patient scapular bone of FIG. 50A;

FIGS. 51A-53B illustrate the top perspective and cutaway side views of the portion of patient scapular bone in FIGS. 50A and 50B during various steps of a surgical technique for using a convertible modular system for shoulder arthroplasty, in accordance with some example embodiments; and

FIG. 54-FIG. 55C illustrate various views of an extraction tool for removing convertible modular systems for shoulder arthroplasty, in accordance with some example embodiments.

### DETAILED DESCRIPTION

The following detailed description and the appended figures are provided to describe and illustrate exemplary embodiments for the purpose of enabling one of ordinary skill in the relevant art to make and use such exemplary embodiments. The description and figures are not intended to limit the scope of the disclosure, or its protection, in any manner.

As used herein the terms “proximal” and “distal” are used to describe opposing axial ends of the particular elements, components, or features being described. The term “attached” refers to the fixed, releasable, or integrated association of two or more elements, components, and/or devices. The term “attached” includes releasably attaching or fixedly attaching two or more elements, components, and/or devices. The terms “medial” and “lateral” are used to describe opposing sides of the particular elements, components, or features being described. The singular forms “a,” “an,” and “the” include plural referents unless the context clearly dictates otherwise.

This disclosure describes several universal or convertible systems or platforms for shoulder arthroplasty. Such systems or platforms are universal or convertible at least in that one or more same components may be utilized for implanting either a glenoid component (e.g., a component having a concave arcuate surface configured to mimic the natural glenoid of the patient, whether such component is implanted into the scapula or the humerus) or a glenosphere component (e.g., a component having a convex outer surface configured to mimic the “ball” portion of the ball-and-socket joint, whether such component is implanted into the humerus or scapula).

During implantation, at least a glenoid or glenosphere component (e.g., an implant component) is ultimately anchored to one of the humerus or scapula of the patient, e.g., patient bone. This disclosure contemplates a variety of ways of accomplishing such anchoring that also provide the desired universality and interconvertibility to ultimately secure either of the glenoid or glenosphere component thereto. General features common to various embodiments, or distinguishing various embodiments, will be described below. Then specific embodiments will be described in more detail in connection with the figures.

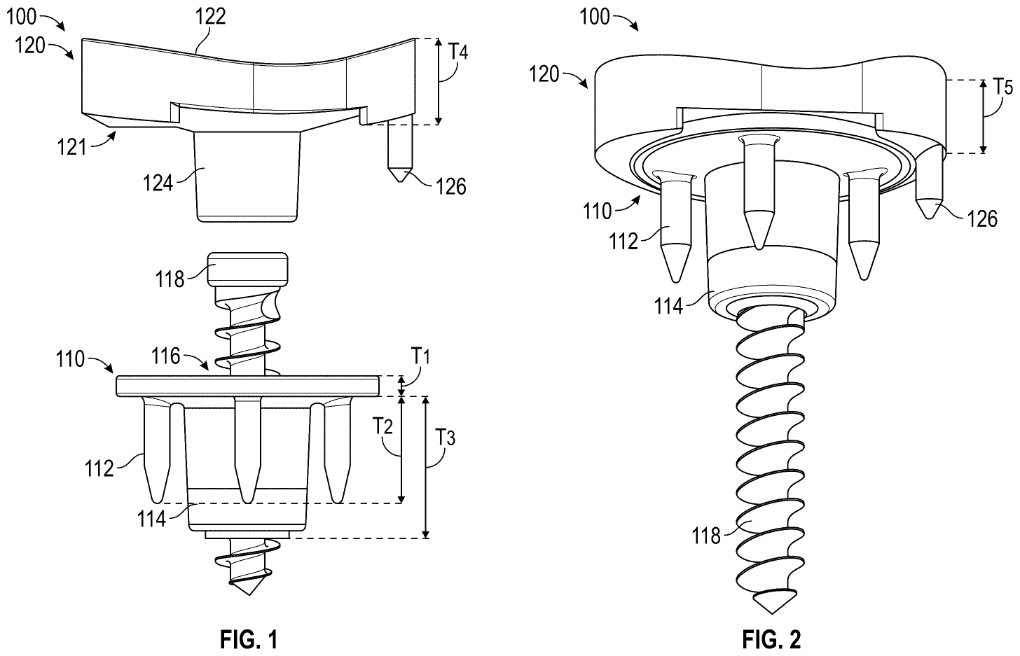

In some embodiments, the implant component is ultimately anchored to bone utilizing a center compression screw (see, e.g., FIGS. 1-8, 21-26B, 35A-35G, 42-45, 47 and 48) that, once properly implanted through a baseplate (see, e.g., FIGS. 1-4, 7, 8, 21-26B, 35A-35G, 42-45 and 48) or through an anchor boss (see, e.g., FIGS. 5, 6 and 47), imparts a compressive force to the baseplate or anchor boss, the baseplate or anchor boss, itself, being coupled and/or coupleable to the respective implant component.

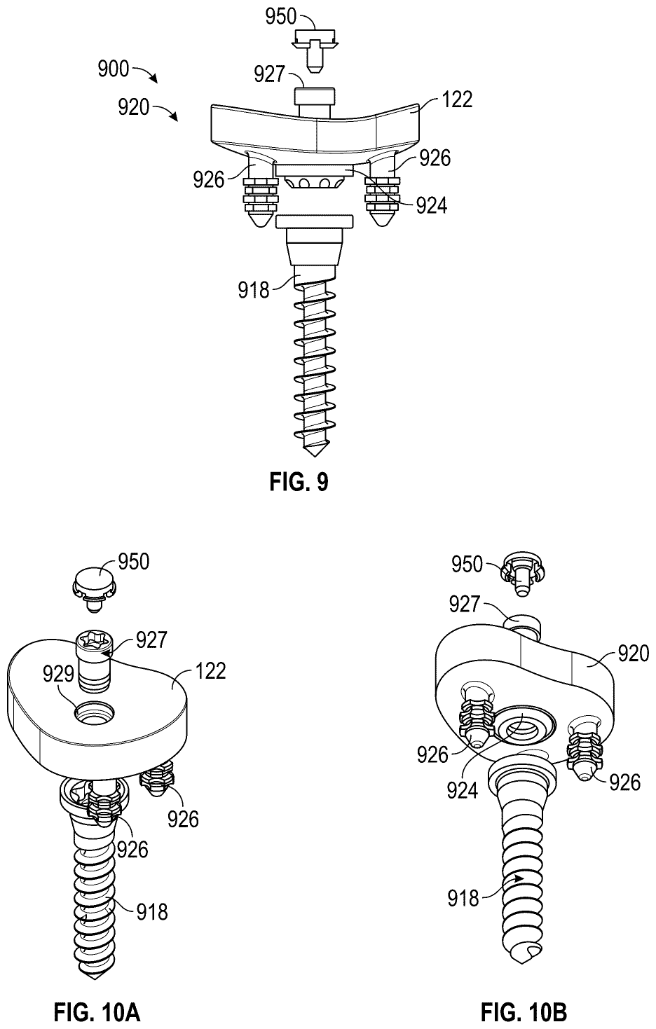

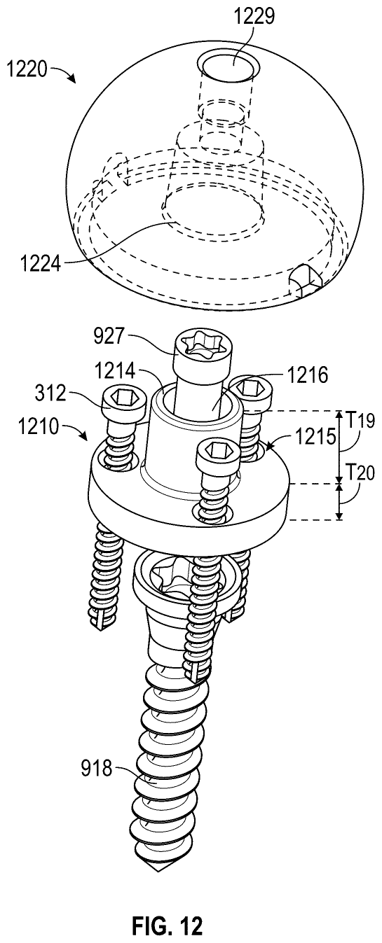

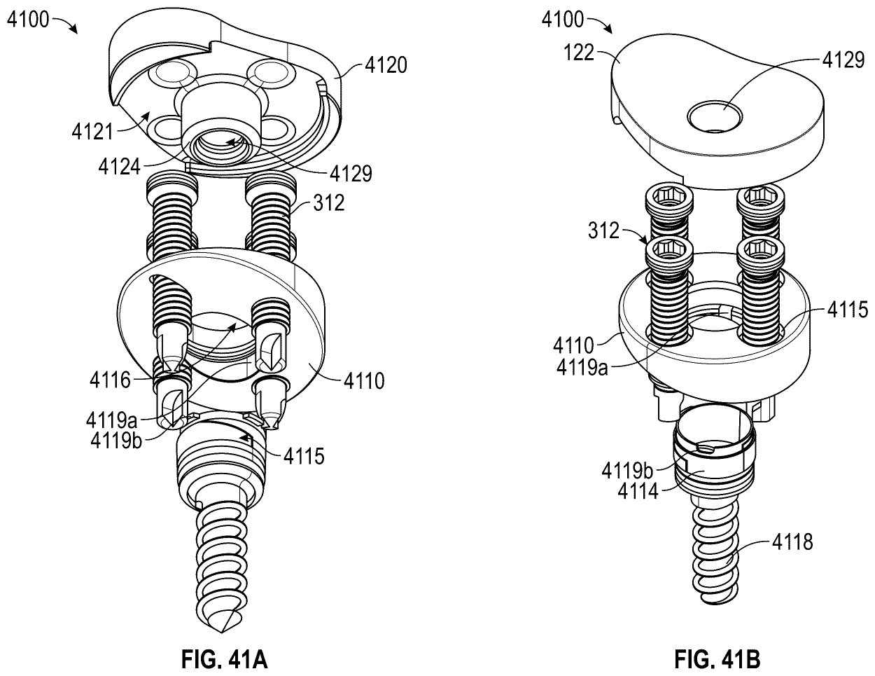

In some other embodiments, the implant component is ultimately anchored to bone utilizing a central anchor screw (see, e.g., FIGS. 9-20) or a bossed central anchor screw (see, e.g., FIGS. 41A, 41B, 46 and 48) that is coupled and/or coupleable, from an underside of the baseplate, to the underside of the baseplate (see, e.g., FIGS. 12, 15-17, 41A, 41B and 48), the baseplate being coupled and/or coupleable to the implant component. In some other embodiments, the central anchor screw is directly coupled or coupleable, from an underside of the implant component, to the underside of the implant component itself (see, e.g., FIGS. 9-11, 13, 14, 18A-19B and 46).

In some embodiments, the central anchor screw is coupled and/or coupleable to a specially-designed metal disk-like component disposed and/or formed on or in the underside of the baseplate (see, e.g., FIGS. 12, 15-17 and 20) or of the implant component itself (see, e.g., FIGS. 9-11, 13, 14 and 18A-19B), the metal disk-like component providing a key interface between the central anchor screw and the baseplate or implant component itself.

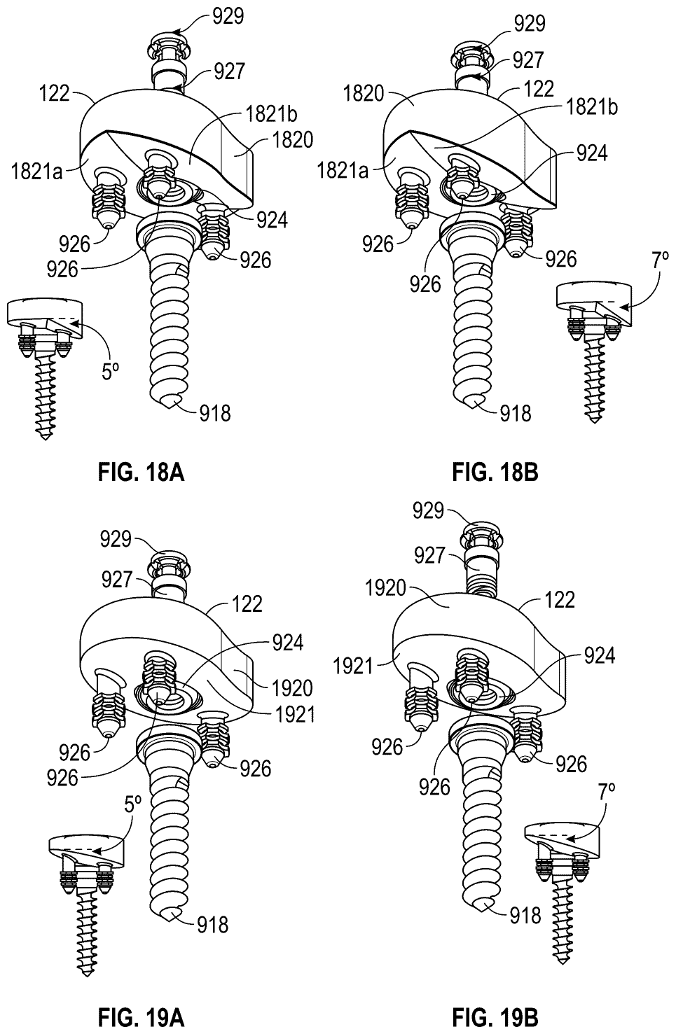

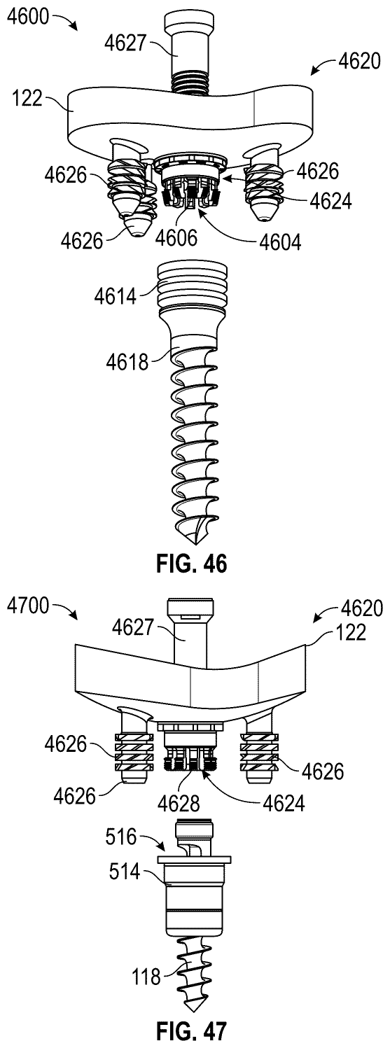

In some embodiments, a baseplate is not utilized (see, e.g., FIGS. 5, 6, 9-11, 13, 14, 18A-19B, 46 and 47). In some such embodiments, a compression screw instead secures an anchor boss into patient bone and the anchor boss is coupled to the implant component (see, e.g., FIGS. 5, 6 and 47). In some other such embodiments, a central anchor screw is implanted into patient bone and the specially-designed metal disk-like component (see, e.g., FIGS. 18A-19B) or a central tapered portion having deflectable extensions disposed on or in the underside of the implant (see, e.g., FIGS. 46 and 47) is coupled to the central anchor screw.

In some other embodiments a baseplate is utilized. In some such embodiments, the baseplate comprises a central bossed portion extending from its underside, which is configured to be disposed within prepared patient bone (see, e.g., FIGS. 1-4, 7, 8, 21-35G, 44 and 45). In some other such embodiments, the baseplate does not comprise a bossed portion extending from its underside but, instead, comprises the specially-designed metal disk-like component disposed on or in its underside, which is configured to couple to a central anchor screw (see, e.g., FIGS. 12, 15-17 and 20). In some other such embodiments, the baseplate comprises neither a central bossed portion extending from its underside nor the specially-designed metal disk-like component, instead, comprising an aperture having a bayonet-type locking feature configured to couple a bossed central anchor screw (see, e.g., FIGS. 41A and 41B). In some other such embodiments, the baseplate comprises a central tapered portion extending from its topside (see, e.g., FIGS. 12, 20 and 48), which, in some cases, is configured to taper or friction fit within a corresponding recess in a glenosphere component. In some embodiments, the baseplate comprises a wedged surface to address particularly-suited patient bone deficiencies and/or abnormalities (see, e.g., FIGS. 18A-20, 35A-35G, 37-41B and 48).

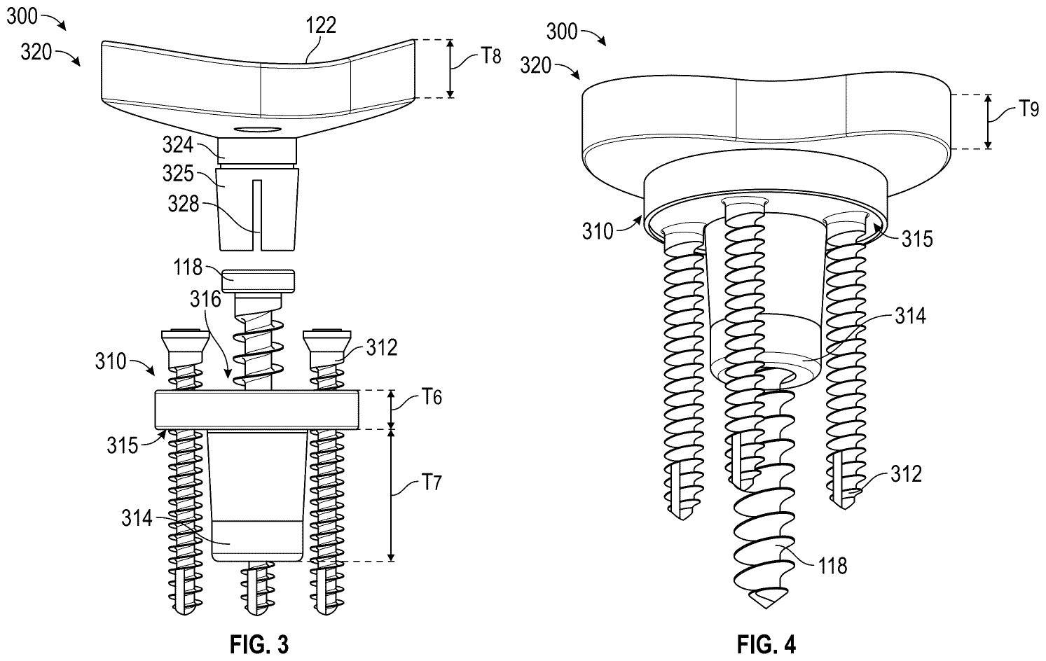

In some embodiments, the baseplate may comprise one or more features to prevent rotation after implantation. In some such embodiments, a plurality of spikes extend from an underside of the baseplate and anchor into bone peripheral to the compression screw (see, e.g., FIGS. 1 and 2). In some other such embodiments, a plurality of rough pegs extend from an underside of the baseplate and anchor into bone peripheral to the compression screw, central anchor screw or bossed central anchor screw (see, e.g., FIGS. 18A-19B, 46 and 47). In some other such embodiments, the baseplate comprises a plurality of peripheral apertures configured to receive peripheral screws therethrough that anchor into bone peripheral to the compression screw, central anchor screw or bossed central anchor screw (see, e.g., FIGS. 3, 4, 7, 8, 12, 15-17, 20-35G, 41-45 and 48).

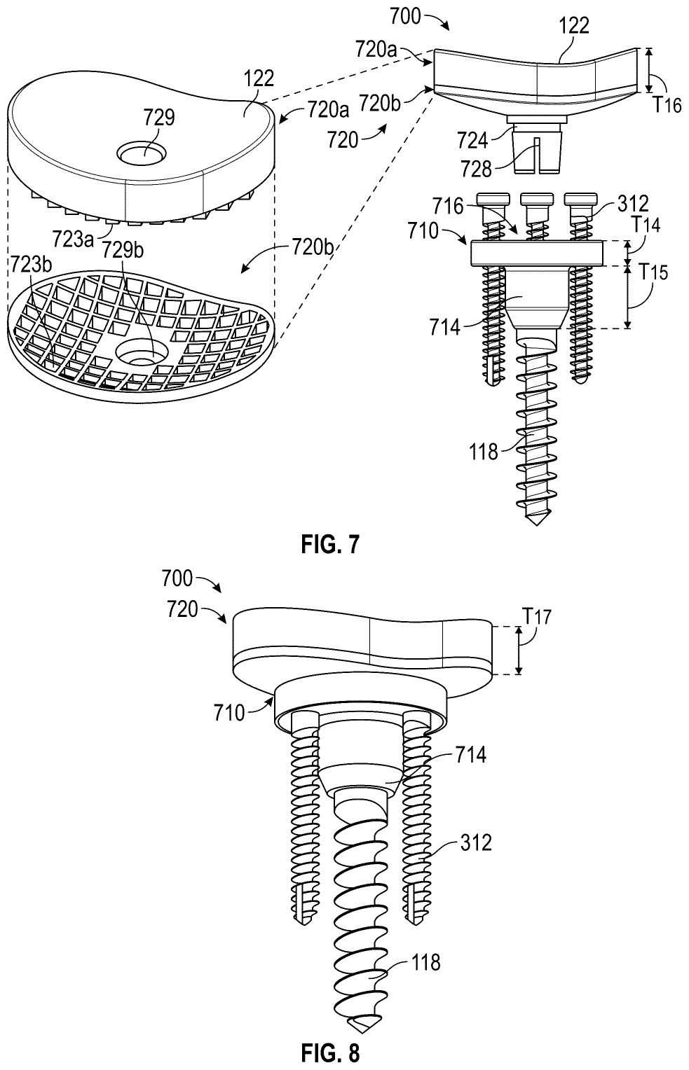

In some embodiments, the implant component comprises a glenoid component (see, e.g., FIGS. 1-11, 13-16E, 18A-19B, 21-22B, 24-26B, 41A, 41B, 44, 46 and 47). In some embodiments, the glenoid component may comprise plastic, in some others metal. In some embodiments, a thickness of the glenoid component at it sulcus (i.e., the lowest point of the arcuate top surface and thinnest part of the glenoid component) may have an example thickness of 4.0-5.0 mm, for example approximately 4.16 mm. However, the disclosure is not so limited and any glenoid component described herein is also contemplated to have any suitable thickness at its sulcus or at any other portion. In yet other embodiments, the glenoid component comprises am upper plastic, e.g., poly, articulation portion and a lower metal portion (see, e.g., FIGS. 7, 8, 13 and 14). In some such embodiments, the glenoid component comprises a central tapered portion extending away from an underside thereof that is configured to taper or friction fit within a recess and/or aperture disposed in a topside of the baseplate. In some embodiments where glenoid component comprises plastic, e.g., poly, a metal tapered extension extends from the central tapered portion and is configured to taper or friction fit within the recess and/or aperture disposed in the topside of the baseplate (see, e.g., FIGS. 3-6). In some embodiments, the glenoid component comprises the specially-designed metal disk-like component disposed on or in its underside to provide a key interface directly with the central anchor screw (see, e.g., FIGS. 9-11, 13, 14 and 18A-19B). In some embodiments, the glenoid component comprises a central tapered portion having the deflectable extensions disposed on or in its underside to provide a key interface directly with the central anchor screw (see, e.g., FIGS. 46 and 47).

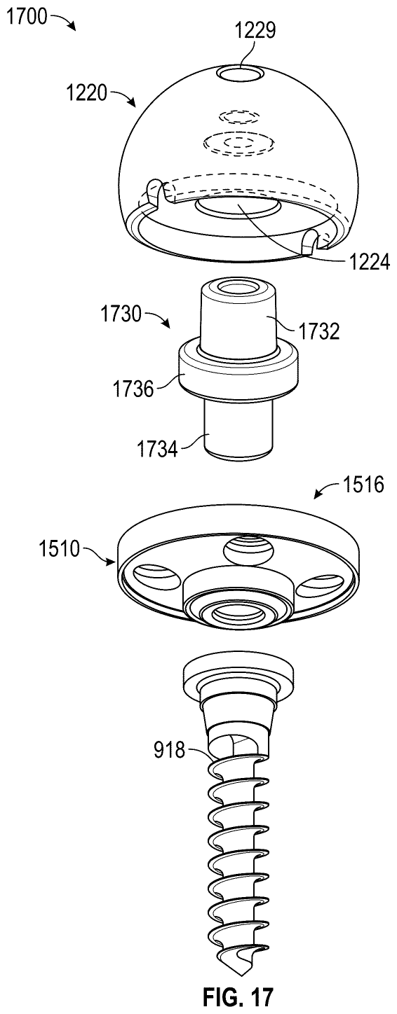

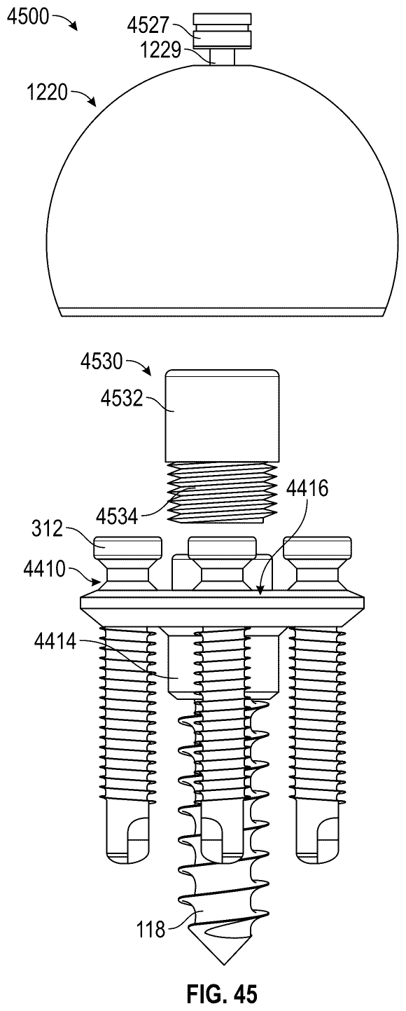

In some embodiments, the implant component comprises a glenosphere component (see, e.g., FIGS. 12, 17, 20, 23A, 23B, 37-40B, 42, 43, 45 and 48), which may comprise a tapered recess configured to taper or friction fit with any of: one tapered end of a dual-taper adapter (the other end of the dual-taper adapter being configured to taper or friction fit within an aperture in a top surface of the baseplate—see, e.g., FIG. 17—or within an aperture of a set screw disposed within the aperture in the top surface of the baseplate; a tapered end of a single-taper, threaded adapter (the other end of the single-taper comprising threads configured to engage with complementary threads in the aperture in the top surface of the baseplate—see, e.g., FIG. 45); or a central tapered portion extending away from a top surface of the baseplate (see, e.g., FIGS. 12, 20 and 48).

Specific embodiments will now be described in connection with the figures.

FIGS. 1 and 2 illustrate an example embodiment of a modular system 100 for shoulder arthroplasty. System 100 comprises a baseplate 110 and a glenoid component 120. In some embodiments, baseplate 110 may be configured for onsetting substantially on a surface of sub-chondral bone.

In some embodiments, baseplate 110 comprises a two-piece assembly including the baseplate 110 itself and a central compression screw 118 configured to be secured through baseplate 110. Central compression screw 118 comprises threads configured to bite into bone of a patient to, thereby, properly align, secure and compress baseplate 110 against and/or within patient bone.

Baseplate 110 comprises a central bossed portion 114 extending from its underside. In some embodiments, central bossed portion 114 has a substantially tapered cylindrical shape. Baseplate 110 comprises a recess and aperture 116 in its top surface. At least a central portion of aperture 116 extends entirely through baseplate 110 such that central compression screw 118 is received through recess 116, extends out through aperture 116 in the underside of baseplate, and a head of central compression screw 118 is entirely disposed within recess 116, below a substantially flat upper surface of baseplate 110 once central compression screw 118 is properly tightened into the bone of the patient.

As illustrated in FIGS. 1 and 2, baseplate 110 may also comprise a plurality of spikes 112 extending away from its underside. Spikes 112 are configured to be disposed into patient bone when central compression screw 118 is received through aperture 116 of baseplate 110 and properly tightened into patient bone and, thereby, prevent undesired rotation of baseplate 110. Such a design is particularly advantageous for reverse shoulder arthroplasty procedures, where baseplate 110 may be secured in a position where there is little available peripheral humeral bone space.

As illustrated in FIG. 1, a top surface of baseplate 110 has a first thickness T1. Spikes 112 are illustrated as extending a length T2 away from an underside of baseplate 110. And central boss 114 is illustrates as extending a length T3 away from the underside of baseplate 110. Example values for T1 include 1.5-2.5 mm, inclusive, for example, 1.84 mm. Example values for T2 include 10.0-12.0 mm, inclusive, for example, 10.41 mm. Example values for T3 include 13.0-15.0 mm, inclusive, for example, 13.73 mm. However, this disclosure is not so limited and T1, T2 and T3 may have any suitable values.

Glenoid component 120 comprises an arcuate top surface 122 configured to directly contact a ball of the patient's humerus (in the case of a partial should arthroplasty), or to a synthetic ball-like replacement component coupled to a portion of the patient's previously prepared humerus (e.g., in the case of total shoulder arthroplasty) or coupled to a portion of the patient's previously prepared scapula (e.g., in the case of total reverse shoulder arthroplasty). Glenoid component 120 comprises a central tapered portion 124 extending from its underside. Central tapered portion 124 is configured to press and taper or friction fit within aperture 116 of baseplate 110 such that the underside of glenoid component 120 is in direct contact with the upper surface of baseplate 110.

In some embodiments, the underside of glenoid component 120 comprises a recessed portion 121 configured to allow at least a portion of baseplate 110 to reside therein when glenoid component 120 is properly coupled to baseplate 110. In some embodiments, glenoid component 120 comprises a peripheral protrusion 126 configured to extend from a peripheral portion of the underside of glenoid component 120 and into a portion of the patient's prepared bone peripheral of baseplate 110. In this way, peripheral protrusion 126 prevents glenoid component 120 from undesirably rotating once glenoid component 120 is locked into baseplate 110.

Glenoid component 120 has a thickness T4 as measured between a topmost peripheral point of arcuate surface 122 and a corresponding bottom-most peripheral point of the underside of glenoid component 120. Example values for T4 include 8.0-9.0 mm, inclusive, for example, 8.56 mm. In some embodiments, system 100 is dimensioned such that an example total thickness T5 of system 100 extending above patient bone is in the range of 7.0-8.0 mm, for example, 7.54 mm.

Glenoid component 120 may comprise a plastic, such as ultra-high molecular weight polyethylene (UHMWPE). However, this disclosure is not so limited and glenoid component 120 may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central compression screw 118 and baseplate 110 may each comprise such a metal or other suitable biocompatible material.

FIGS. 3 and 4 illustrate another example embodiment of a modular system 300 for shoulder arthroplasty. System 300 comprises a baseplate 310 and a glenoid component 320. In some such embodiments, baseplate 310 may be configured for insetting below sub-chondral bone.

In some embodiments, baseplate 310 is also two-piece assembly comprising the baseplate 310 itself and central compression screw 118, configured to properly align, secure and compress baseplate 310 against and/or within patient bone.

Baseplate 310 comprises a central bossed portion 314 extending from its underside. In some embodiments, central bossed portion 314 has a substantially tapered cylindrical shape. Baseplate 310 comprises a recess and aperture 316 in its top surface. At least a central portion of aperture 316 extends entirely through baseplate 310 such that central compression screw 118 is received through recess 316, extends out through aperture 316 in the underside of baseplate, and a head of central compression screw 118 is entirely disposed within recess 316, below a substantially flat upper surface of baseplate 310 once central compression screw 118 is properly tightened into the bone of the patient.

As illustrated in FIGS. 3 and 4, rather than utilizing spikes 112, baseplate 310 comprises a plurality of peripheral apertures 315 each receiving one of a plurality of peripheral screws 312 comprising threads configured to bite into patient bone peripheral of central compression screw 118 and, thereby, prevent undesired rotation of baseplate 310. In some embodiments, peripheral screws 312 have a head configured for Torx drive, e.g., Torx bits. In some embodiments, the teeth of peripheral screws 312 have a 5 mm diameter. In some embodiments, peripheral screws 312 have a length of 14 mm, 18 mm, 22 mm, 26 mm, 30 mm, 34 mm, 38 mm or any other suitable length. In some embodiments, longer peripheral screws 312 help to address applications where there is advanced patient bone loss.

As illustrated in FIG. 3, a top surface of baseplate 310 has a thickness T6. Example values for T6 include 3.5-4.5 mm, inclusive, for example, 4.02 mm. And central boss 314 is illustrated as extending a length T7 away from the underside of baseplate 310. Example values for T7 include 13.0-14.0 mm, inclusive, for example, 13.44 mm. However, this disclosure is not so limited and T6 and T7 may have any suitable values.

Glenoid component 320 comprises arcuate top surface 122 as previously described for glenoid component 120 in FIGS. 1 and 2. Glenoid component 320 comprises a central tapered portion 324 extending from its underside. However, in contrast to the embodiments shown in FIGS. 1 and 2, central tapered portion 324 extends only a relatively short (e.g., a smaller or shorter) distance away from the underside of glenoid component 320 (e.g., compared to central tapered portion 124 of FIG. 1) and a metal tapered extension (or collet) 325 is coupled to a distal end of central tapered portion 324, for example via complementary threads disposed on mating ends of metal tapered extension 325 and central tapered portion 324. Metal tapered extension 325 is configured to press and taper or friction fit within aperture 316 of baseplate 310 such that the underside of glenoid component 320 is in direct contact with the upper surface of baseplate 310. In some embodiments, metal tapered extension 325 comprises one or more vertically oriented slots 328 extending from a distal end of metal tapered extension 325. Vertically oriented slot(s) 328 may allow metal tapered extension 325 to deform slightly enough to progress sufficiently into recess 316 of baseplate 310 and provide a satisfactory metal-on-metal taper or friction lock between metal tapered extension 325 and the inner surface of recess 316.

Glenoid component 320 has a thickness T8 as measured between a topmost peripheral point of arcuate surface 122 and a corresponding bottom-most peripheral point of the underside of glenoid component 320. Example values for T8 include 5.0-7.0 mm, inclusive, for example, 5.87 mm. In some embodiments, system 300 is dimensioned such that a total thickness T9 of system 100 extending above patient bone is approximately 4.2 mm. Example values for T9 include 5.0-7.0 mm, inclusive, for example, 5.87 mm.

Glenoid component 320 may comprise a plastic, such as UHMWPE. However, this disclosure is not so limited and glenoid component 320 may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central compression screw 318, metal tapered extension 325 and baseplate 310 may each comprise such a metal or other suitable biocompatible material.

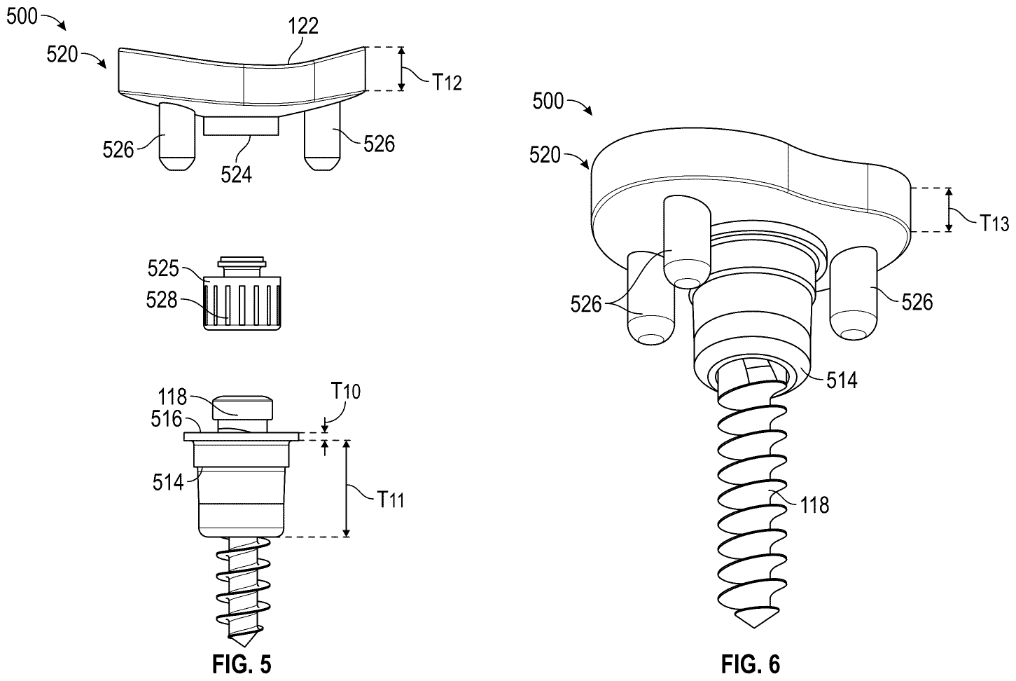

FIGS. 5 and 6 illustrate yet another example embodiment of a modular system 500 for shoulder arthroplasty. In contrast to the example embodiments of FIGS. 1-4, system 500 comprises an anchor boss 514 rather than a baseplate. In some such embodiments, anchor boss 514 may be configured for onsetting substantially on a surface of sub-chondral bone.

In some embodiments, anchor boss 514 may be substantially similar to the central bossed portions 114, 314 of respective baseplates 110, 310. For example, anchor boss 514 has a substantially tapered cylindrical shape and comprises a recess and aperture 316 in its top surface. At least a central portion of aperture 516 extends entirely through anchor boss 514 such that central compression screw 118 is received through recess 516, extends out through aperture 516 in the underside of anchor boss 514, and a head of central compression screw 118 is entirely disposed within recess 516, below a substantially flat upper surface of anchor boss 514 once central compression screw 118 is properly tightened into the bone of the patient.

As illustrated by FIGS. 5 and 6, anchor boss 514 is not a baseplate, does not have a top surface that appreciably extend radially away from aperture 316 and, itself, does not comprise peripheral screws, spikes or pegs for securing to peripheral patient bone. Such a design is particularly advantageous for reverse shoulder arthroplasty procedures, where anchor boss 514 may be secured where little peripheral humeral bone space is available.

As illustrated in FIG. 5, a top surface (e.g., lip) of anchor boss 514 has a thickness T10. Example values for T10 include 1.0-2.0 mm, inclusive, for example, 1.02 mm. And anchor boss 514 is illustrates as extending a length T11 below an underside of this top surface (e.g., lip). Example values for T11 include 12.0-13.0 mm, inclusive, for example, 12.55 mm. However, this disclosure is not so limited and T10 and T11 may have any suitable values. In some embodiments, anchor boss 514 is configured to accommodate all-poly thicknesses.

Glenoid component 520 comprises arcuate top surface 122 as previously described. Glenoid component 520 comprises a central tapered portion 524 extending from its underside. However, in contrast to the embodiments shown in FIGS. 1 and 2, central tapered portion 524 extends only a relatively short (e.g., a smaller or shorter) distance away from the underside of glenoid component 520 (e.g., compared to central tapered portion 124 of FIG. 1) and a metal tapered extension (or collet) 525 is coupled to a distal end of central tapered portion 524, for example via complementary threads disposed on mating ends of metal tapered extension 525 and central tapered portion 524. Metal tapered extension 525 is configured to press and taper or friction fit within aperture 516 of anchor boss 514 such that the underside of glenoid component 520 is in direct contact with the upper surface of anchor boss 514. In some embodiments, metal tapered extension 525 comprises one or more vertically oriented slots or grooves 528 extending from a distal end of metal tapered extension 525. In some embodiments, such vertically oriented slots or grooves 528 may allow metal tapered extension 525 to deform slightly enough to progress sufficiently into recess 516 of anchor boss 514 and provide a satisfactory metal-on-metal taper or friction lock between metal tapered extension 525 and the inner surface of recess 516. In some embodiments, such vertically oriented slots or grooves 528 provide edges on metal tapered extension 525 that provide a more robust taper or frictional fit with the inner surface of recess 516.

In some embodiments, glenoid component 520 comprises a plurality of peripheral protrusions 526 configured to extend from a peripheral portion of the underside of glenoid component 520 and into a portion of the patient's prepared bone peripheral of anchor boss 514. In this way, peripheral protrusions 526 prevent glenoid component 520 from undesirably rotating once pressed and locked into anchor boss 514.

Accordingly, central compression screw 118 locks down anchor boss 514 and metal tapered extension 525 locks glenoid component 520 to anchor boss 514. In some embodiments, for example some TSA cases, anchor boss 514 accommodates tri-lobe poly legs (not shown).

Glenoid component 520 has a thickness T12 as measured between a topmost peripheral point of arcuate surface 122 and a corresponding bottom-most peripheral point of the underside of glenoid component 520. In some embodiments, system 500 is dimensioned such that a total thickness T13 of system 100 extending above patient bone is approximately 4.2 mm.

Glenoid component 520 may comprise a plastic, such as UHMWPE. However, this disclosure is not so limited and glenoid component 520 may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central compression screw 118, metal tapered extension 525 and anchor boss 514 may each comprise such a metal or other suitable biocompatible material.

FIGS. 7 and 8 illustrate yet another example embodiment of a modular system 700 for shoulder arthroplasty. System 700 comprises a baseplate 710 and a glenoid component 720. In some such embodiments, baseplate 710 may be configured for insetting below sub-chondral bone.

In some embodiments, baseplate 710 comprises a two-piece assembly including the baseplate 710 itself and central compression screw 118, configured to properly align, secure and compress baseplate 710 against and/or within patient bone. Baseplate 710 also comprises a plurality of peripheral apertures 715 each receiving one of a plurality of peripheral screws 312 for peripherally securing baseplate 710 to patient bone, as previously described.

Baseplate 710 comprises a central bossed portion 714 extending from its underside. In some embodiments, central bossed portion 714 may be substantially as previously described for central bossed portion 314 of FIGS. 3 and 4, having a substantially tapered cylindrical shape, comprising a recess and aperture 716 in its top surface, at least a portion of which extension entirely through central bossed portion 714 and baseplate 710, and configured to receive central compression screw 118 therethrough. Central bossed portion 714 may be coated with, or formed to have, a porous metal layer on at least some of its bone-facing surface. Such a porous metal layer allows superior, cement-less anchoring of baseplate 710 to patient bone. Example porosities for porous metal layer include but are not limited to an average porosity of 65%. In some cases, multiple (e.g., 2) pore sizes may be utilized. In some cases, such a porous metal layer may comprise a titanium non-spherical bead porous coating. Such coatings may conform to ASTM F67. Such coatings may be applied to, for example, titanium substrate devices. An example thickness of such a porous metal layer is approximately 1.5 mm. However, this disclosure is not so limited and any suitable thickness is also contemplated.

In some embodiments, central compression screw 118 may alternatively be an integral part of baseplate 710 such that baseplate 710 rotates with central compression screw 118 when central compression screw 118 is rotated.

As illustrated in FIG. 7, a top surface of baseplate 710 has a thickness T14. Example values for T14 include 3.5-4.5 mm, inclusive, for example, 4.02 mm. And central boss 714 is illustrates as extending a length T15 away from the underside of baseplate 710. Example values for T15 include 9.0-10.0 mm, inclusive, for example, 9.53 mm. However, this disclosure is not so limited and T14 and T15 may have any suitable values.

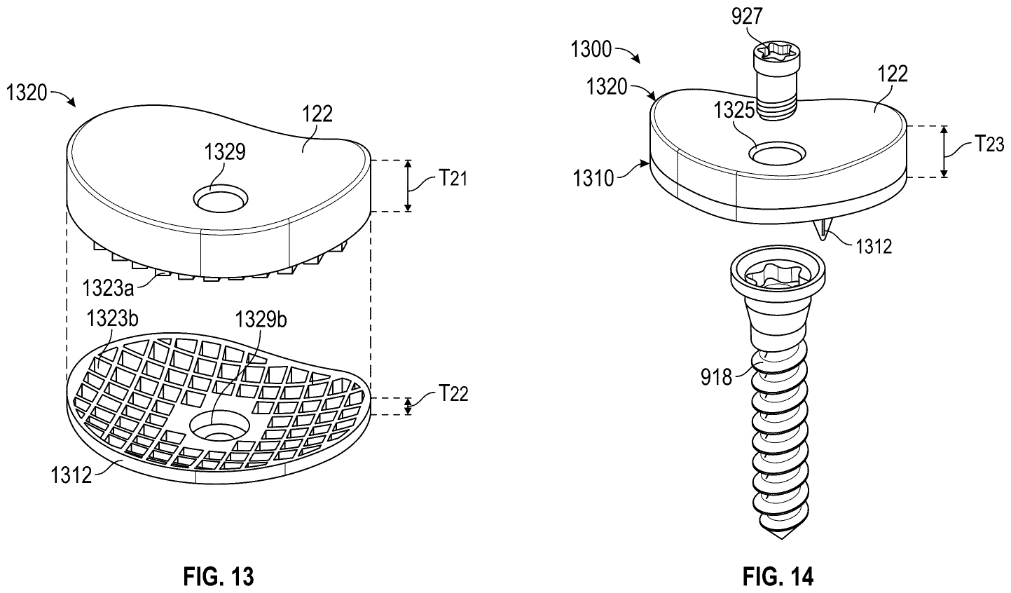

In some embodiments, glenoid component 720 is also a two-piece assembly comprising an upper portion 720a and a lower portion 720b configured to mate with upper portion 720a. Upper portion 720a comprises arcuate top surface 122 as previously described. An aperture 1325 may also be disposed in top surface 122, configured to receive an implant locking screw (not shown, but see, e.g., 1327 in FIG. 13) for securing assembled glenoid component 720 to baseplate 710. Upper portion 720a may also comprise a patterned surface 723a on its underside (e.g., a waffle-like pattern of raised elements having any suitable cross-section, for example square, rectangular, circular, ovoid, or regularly or irregularly polygonal).

Lower portion 720b also comprises a patterned top surface 723b having a complementary shape to patterned bottom surface 723a of upper portion 720a. An aperture 729 also passes through lower portion 720b and is disposed such that when upper portion 720a and lower portion 720b are fitted together, apertures 729 and 729b align for receiving the implant locking screw. Lower portion 720b also comprises a central tapered portion 724 extending from its underside. In some embodiments, central tapered portion 724 is an integrally-formed part of lower portion 720b. In other embodiments, at least a portion of central tapered portion 724 is coupled to lower portion 720b similarly to metal tapered extension 325 in FIGS. 3 and 4, for example via complementary threads disposed on mating ends of central tapered portion 724 and lower portion 720b. Central tapered portion 724 is configured to press and taper or friction fit within aperture 716 of baseplate 710 such that the underside of glenoid component 720 is in direct contact with the upper surface of baseplate 710. In some embodiments, central tapered portion 724 comprises one or more vertically oriented slots 728 extending from a distal end of central tapered portion 724. Vertically oriented slot(s) 728 may allow central tapered portion 724 to deform slightly enough to progress sufficiently into recess 716 of baseplate 710 and provide a satisfactory metal-on-metal taper or friction lock between central tapered portion 724 and the inner surface of recess 716.

Glenoid component 720 has a combined thickness T16 (i.e., a thickness of upper portion 720a and lower portion 720b when properly assembled) measured between a topmost peripheral point of arcuate surface 122 and a corresponding bottom-most peripheral point of the underside of lower portion 720b of glenoid component 720. Example values for T16 include 6.5-7.5 mm, inclusive, for example, 6.91 mm. In some embodiments, system 700 is dimensioned such that a total thickness T17 of system 700 extending above patient bone is approximately 6.5-7.5 mm, inclusive, for example, 6.91 mm.

Upper portion 720a of glenoid component 720 may comprise a plastic, such as UHMWPE. However, this disclosure is not so limited and upper portion 720a may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central compression screw 118, lower portion 720b of glenoid component 720, central tapered portion 724 (where detachable), central compression screw 118 and baseplate 710 may each comprise such a metal or other suitable biocompatible material.

FIGS. 9-11 illustrate yet another example embodiment of a modular system 900 for shoulder arthroplasty. System 900 comprises a glenoid component 920 and a central anchor screw 918. System 900 does not include a baseplate. Instead, anchor screw 918 comprises threads configured to bite into bone of a patient and provide a stand-alone anchor for properly aligning and securing glenoid component 920 directly thereto and against and/or within patient bone, as will be described in more detail below. Importantly, in embodiments according to FIGS. 9-11, the stand-alone anchoring property of central anchor screw 918 allows the potential to eliminate the baseplate. And this reduces part count, attendant practitioner confusion, and the potential for making a mistake during a patient procedure.

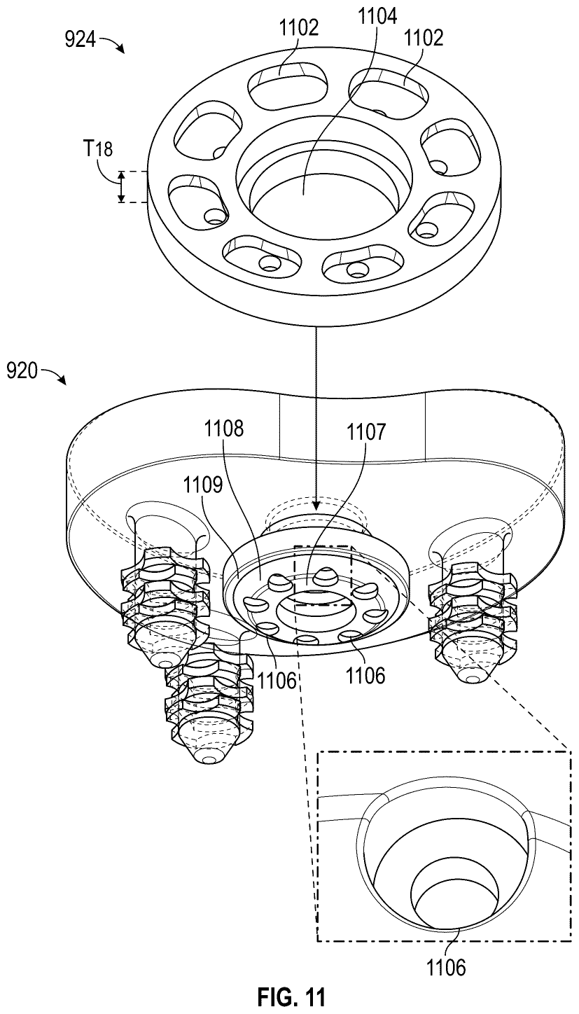

In some embodiments, glenoid component 920 is a multi-piece assembly. For example, comprising arcuate top surface 122, having an aperture 929 disposed therein, and a metal disk-like component 924 disposed against or molded into a bottom surface of glenoid component 920. Metal disk-like component 924 provides a key locking interface between glenoid component 920 and central anchor screw 918, as will be described in more detail in connection with FIG. 11 below.

Implant locking screw 927 is configured to pass and/or thread through aperture 929 and engage with complementary threads of a head of central anchor screw 918 and, in some cases, threads within aperture 1104 of metal disk-like component 924. In some embodiments, a screw cap cover 950 is configured to fit within aperture 929 in arcuate top surface 122 of glenoid component 920 and over implant locking screw 927 to, thereby, ensure a substantially continuous and smooth transition between immediately adjacent edges of arcuate top surface 122 and screw cap cover 950.

Glenoid component 920 comprises a plurality of rough peripheral protrusions 926 configured to extend from a peripheral portion of the underside of glenoid component 920 and into a portion of the patient's prepared bone peripheral of central anchor screw 918. In this way, rough peripheral protrusions 926 help to prevent glenoid component 920 from undesirably rotating once locked into central anchor screw 918.

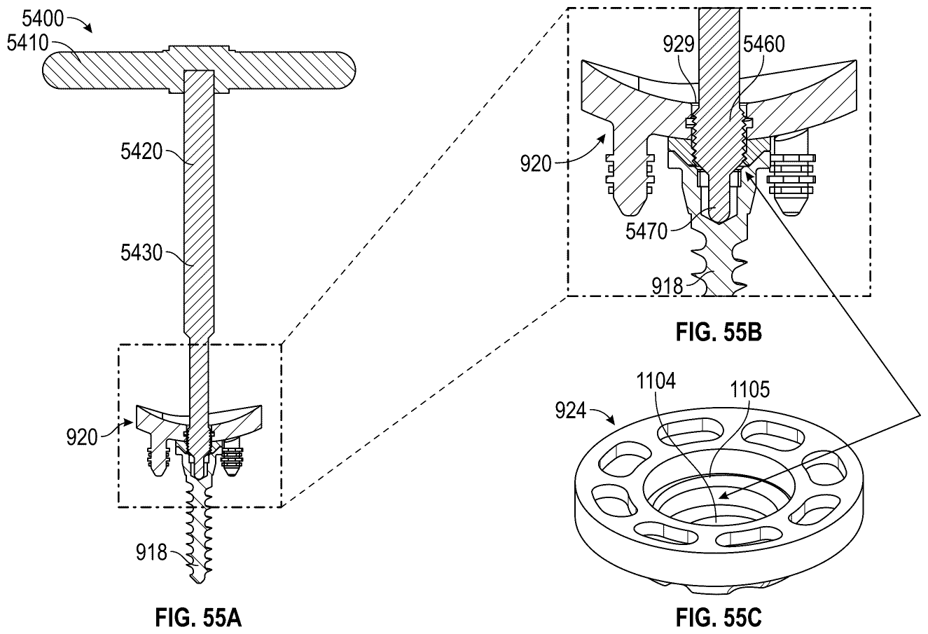

As shown in FIG. 11, metal disk-like component 924 has a substantially circular form factor and a thickness T18. Metal disk-like component 924 comprises a central aperture 1104 into which at least a portion of implant locking screw 927 may thread or at least pass through. While not illustrated in FIG. 11, an inner surface of central aperture 1104 may comprise such threads (see, e.g., FIG. 55C). In some embodiments, central aperture 1104 has a substantially circular form factor. A top surface of metal disk-like component 924 is illustrated as comprising a plurality of recesses 1102. In some embodiments, each of recesses 1102 may have a form factor comprising a different portion of a same circular track and having rounded ends. A plurality of thru-holes 1106 may be c-bored from a bottom surface of metal disk-like component 924. C-boring may comprise boring a hole into and/or through an at least partially angled portion of the bottom surface of metal disk-like component 924 such that the bottom-most portions of the sidewalls of the thru-holes 1106 extend partially around each thru-hole and taper away toward central aperture 1104 (e.g., into a “C” shape when viewed from above or below). For example, as shown in FIG. 11, the bottom surface of metal disk-like component 924 may comprise an innermost portion 1107 peripheral to aperture 1104, an outermost portion 1109 along the periphery of the bottom surface of metal disk-like component 924, and an intermediate portion 1108 extending between the innermost 1107 and outermost 1109 portions. In some embodiments, innermost portion 1107 may extend along a first plane, outermost portion 1109 may extend along a second plane different from the first plane, and intermediate portion 1108 may extend or slope between the first and second planes and from the first toward the second plane, or vice versa. Thru-holes 1106 may be bored in positions such that a portion of each thru-hole 1106 extends through a part of innermost portion 1107 and through an immediately adjacent part of intermediate portion 1108. Such C-boring allows mating features in the head of central anchor screw 918 to mechanically engage with the outer portions of these sidewalls. In some embodiments, thru-holes 1106 extend through to respective ones of the plurality of recesses 1102.

In some embodiments where glenoid component 920 comprises a plastic, metal disk-like component 924 may be molded or overmolded into the bottom surface of glenoid component 920. In some other embodiments, for example where glenoid component 920 comprises a metal, metal disk-like component 924 may be machined into or as an integral part of glenoid component 920 (e.g., omitting the one or more recesses 1120 in a top surface, since such a “top surface” would no longer be an outer surface but at a position internal to glenoid component 920).

In some embodiments, glenoid component 920 may have a similar thickness to that of some prior glenoid component embodiments, e.g., 4.2 mm, as measured between a topmost peripheral point of arcuate surface 122 and a corresponding bottom-most peripheral point of the underside of glenoid component 920. In some embodiments, system 900 is dimensioned such that a total thickness of system 900 that extends above the prepared bone into which central anchor screw 918 is secured is approximately 4.2 mm.

Glenoid component 720 may comprise a plastic, such as UHMWPE. However, this disclosure is not so limited and upper portion 720a may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central anchor screw 918 and disk-like component 924 may each comprise such a metal or other suitable biocompatible material.

FIG. 12 illustrates yet another example embodiment of a modular system 1200 for shoulder arthroplasty. System 1200 comprises a baseplate 1210 and a glenosphere component 1220. In some embodiments, baseplate 1210 may be configured for onsetting substantially on, rather than below, a surface of sub-chondral bone.

In some such embodiments, baseplate 1210 is at least a two-piece assembly comprising the baseplate 1210 itself and central anchor screw 918 as previously described. While not visible in FIG. 12, an underside of baseplate 1210 comprises at least the bottom portion of metal disk-like component 924 as previously described. In some embodiments, rather than being molded or overmolded into a poly glenoid component, metal disk-like component 924 may be machined into or as an integral part of baseplate 1210 (e.g., omitting the one or more recesses 1120 in a top surface, since such a “top surface” would no longer be an outer surface but at a position internal to baseplate 1210).

Baseplate 1210 comprises a central tapered portion 1214 extending away from a top surface of baseplate 1210. Central tapered portion 1214 has a substantially tapered cylindrical shape. Central tapered portion 1214 comprises a recess and aperture 1216 in its top surface. At least a central portion of aperture 1216 extends entirely through baseplate 1210 such that an implant locking screw 927 is received through recess 1216, extends out through aperture 1216 in the underside of baseplate, and its thread engage with complementary threads of the head of central anchor screw 918 and, in some cases, threads within aperture 1104 of metal disk-like component 924.

Baseplate 1210 also comprises a plurality of peripheral apertures 1215 each receiving a peripheral screw 312 configured to prevent undesired rotation of baseplate 1210, as previously described. Baseplate 1210 has a thickness T20 of approximately 4.0-5.0 mm, inclusive, for example 4.27 mm. And central tapered portion 1214 extends for a distance T19 of approximately 8.5-9.5 mm, inclusive, for example 8.89 mm.

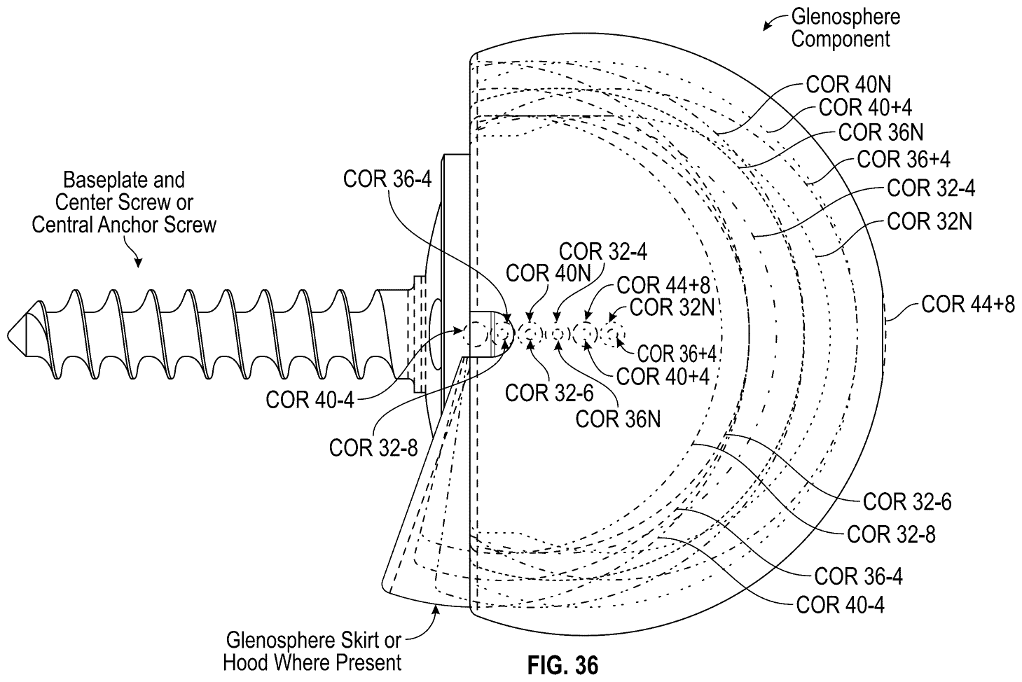

Glenosphere component 1200 has a substantially convex shape configured to substantially replicate or simulate the “ball” of the ball and socket joint of the shoulder. This disclosure contemplates several options for glenosphere component 1220, some of which are illustrated in FIG. 36. For example, and not limitation, the convex portion of glenosphere 1220 may have a diameter and/or proportional radius of curvature of 32 mm, 36 mm, 40 mm or 44 mm. In some embodiments, 32 mm options have 2 mm of lateral offset, see e.g., COR 36-6 in FIG. 36. In some embodiments, 32 mm options have 4 mm of lateral offset, see e.g., COR 36-8 in FIG. 36. In some embodiments, 36 mm options have 10 mm of lateral offset, see e.g., COR 36+4 in FIG. 36. In some embodiments, 40 mm options have 8 mm of lateral offset, see e.g., COR 40+4 in FIG. 36. In some embodiments, a 40N sphere option has a hood large enough to cover a baseplate wedge (see, e.g., baseplate wedge 1910 of FIG. 19), e.g., having a 4 mm offset, see e.g., COR 40N in FIG. 36.

Glenosphere component 1220 comprises a recess 1224 in its bottom side configured to receive and friction fit with central tapered portion 1214. Accordingly, in some embodiments, recess 1224 has a complementary taper to that of central tapered portion 1214 of baseplate 1210. In some embodiments, glenosphere component 1220 comprises an aperture 1229 disposed opposite recess 1224 and configured to receive an implant locking screw (not shown but see, e.g., 927). Such an implant locking screw may be configured to secure glenosphere component 1220 to baseplate 1210, for example, having distal thread configured to engage with complementary threads in at least one of a head of implant locking screw 927 (once secured within aperture 1216), a portion of baseplate 1210 (e.g., within aperture 1216 of central tapered portion 1214), and/or a head of central anchor screw 918 (where such implant locking screw 927 is not utilized and this implant locking screw is sufficiently long to seat in aperture 1229 while passing through glenosphere component 1220, aperture 1216 in central tapered portion 1214 and its threads engage with complementary threads of one or both of metal disk-like component 924 and the head of central anchor screw 918, as previously described for implant locking screw 927). Any or all such combinations are contemplated.

Glenosphere component 1220 may comprise a plastic, such as UHMWPE. However, this disclosure is not so limited and glenosphere component 1220 may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central anchor screw 918, baseplate, and peripheral screws 312 may each comprise such a metal or other suitable biocompatible material.

FIGS. 13-14 illustrate yet another example embodiment of a modular system 1300 for shoulder arthroplasty. System 1300 incorporates features of the embodiments shown in at least FIGS. 1-2, FIG. 5-8 and FIGS. 9-11. For example, system 1300 comprises central anchor screw 918 as previously described and a glenoid component 920. Accordingly, system 1300 does not include a baseplate in the sense previously shown in FIGS. 1-4 and 7-8. Glenoid component 1320 is also illustrated as a two-piece assembly comprising an upper portion 1320a and a lower portion 1320b configured to mate with upper portion 1320a. Upper portion 1320a comprises arcuate top surface 122 as previously described. An aperture 1325 may also be disposed in top surface 122, configured to receive implant locking screw 927. Upper portion 1320a may also comprise a patterned surface 1323a on its underside (e.g., a waffle-like pattern of raised elements having any suitable cross-section, for example square, rectangular, circular, ovoid, or regularly or irregularly polygonal).

Lower portion 1320b also comprises a patterned top surface 1323b having a complementary shape to patterned bottom surface 1323a of upper portion 1320a. An aperture 1329b also passes through lower portion 1320b and is disposed such that when upper portion 1320a and lower portion 1320b are fitted together, apertures 1329 and 1329b align for receiving implant locking screw 927. While not visible in FIGS. 13-14, an underside of lower portion 1320b of glenoid component 1320 comprises at least the bottom portion of metal disk-like component 924 as previously described. Accordingly, threads of implant locking screw 927 may be configured to engage with complementary threads of one or both of metal disk-like component 924 and a head of central anchor screw 918. Lower portion 1320b may also comprise a plurality of spikes 1312 extending away from its underside. Spikes 1312 are configured to push into patient bone when glenoid component 1320 is coupled to central anchor screw 918 via implant locking screw 927 and metal disk-like component 924.

Upper portion 1320a may have a thickness T21 of approximately 5.0-6.0 mm, inclusive, for example 5.13 mm. And lower portion 1320b may have a thickness T22 of approximately 2.0-3.0 mm, inclusive, for example 2.08 mm. Accordingly, glenoid component 1320 has a combined thickness T23 (i.e., a thickness of upper portion 1320a and lower portion 1320b when properly assembled) similar to that of some other embodiments of a glenoid component described herein, e.g., 7.0-8.0 mm, inclusive, for example 7.23 mm as measured between a topmost peripheral point of arcuate surface 122 and a corresponding bottom-most peripheral point of the underside of lower portion 1320b of glenoid component 1320.

Upper portion 1320a of glenoid component 1320 may comprise a plastic, such as UHMWPE. However, this disclosure is not so limited and upper portion 1320a may also or alternatively comprise a metal, or other suitable biocompatible material, e.g., titanium (Ti) and/or cobalt alloys (e.g., cobalt-chromium (CoCr), cobalt-chromium-molybdenum (CoCrMo)). Central anchor screw 918 and lower portion 1320b of glenoid component 1320 may each comprise such a metal or other suitable biocompatible material.

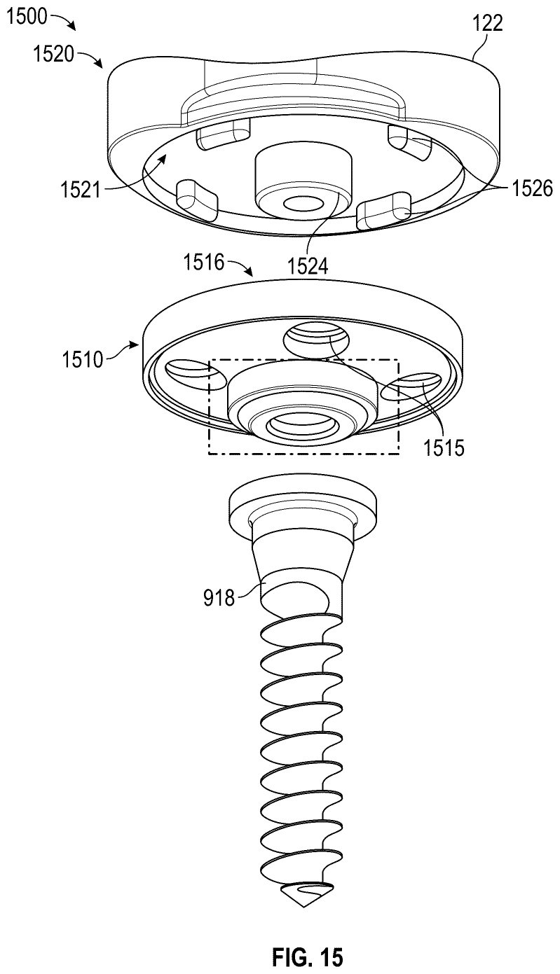

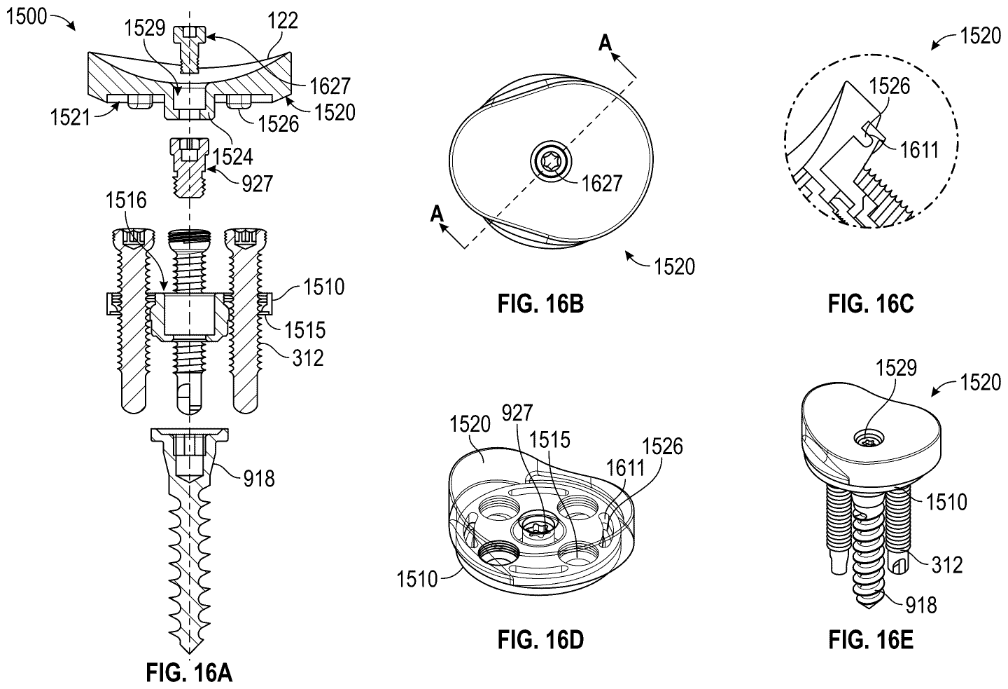

FIGS. 15 and 16A-16E illustrate yet another example embodiment of a modular system 1500 for shoulder arthroplasty. System 1500 comprises central anchor screw 918 as previously described, a baseplate 1510 and a glenoid component 1520. In some embodiments, system 1500 may be configured for onsetting substantially on a surface of sub-chondral bone.

Similar to some previously described embodiments, baseplate 1510 may be considered a two-piece assembly comprising a metal baseplate 1510 and central anchor screw 918. As can be seen in the white dotted-lined box in FIG. 15, an underside of baseplate 1510 comprises at least the bottom portion of metal disk-like component 924, machined into or as an integral part of metal baseplate 1510, also as previously described. Baseplate 1510 comprises a recess and aperture 1516 in its top surface. At least a central portion of aperture 1516 extends entirely through baseplate 1510 such implant locking screw 927 is configured to pass and/or thread through aperture 1516 and engage with complementary threads of one or both of metal disk-like component 924 (e.g., the bottom-most portion of aperture 1516) and a head of central anchor screw 918. Baseplate 1510 also comprises a plurality of peripheral apertures 1515 each receiving a peripheral screw 312 substantially as previously described.

Glenoid component 1520 comprises arcuate top surface 122 as previously described. An aperture 1529 is disposed in arcuate top surface 122 and configured to receive implant locking screw 1627 configured to secure glenoid component 1520 to baseplate 1510 by engaging its threads with complementary threads in at least one of baseplate 1510 (e.g., in an inner wall of the top portion of aperture 1516) and/or the head of implant locking screw 927 securing baseplate 1510 to central anchor screw 918 below.

Glenoid component 1520 comprises a central tapered portion 1524 extending from its underside. Central tapered portion 1524 is configured to press and taper or friction fit within the upper portion of aperture 1516 of baseplate 1510 such that the underside of glenoid component 1520 is in direct contact with the upper surface of baseplate 1510. In some embodiments, the underside of glenoid component 1520 comprises a recessed portion 1521 configured to allow at least a portion of baseplate 1510 to reside therein when glenoid component 1520 is properly coupled to baseplate 1510. Glenoid component 1520 may also comprise a plurality of snap-in features (e.g., tapered ribs) 1526 configured to snap and/or friction fit into proper alignment and couple with mating features 1611 disposed in a topside of baseplate 1510 when glenoid component 1520 is pressed into baseplate 1510 with sufficient force. In some embodiments, mating features 1611 may comprise discontinuous portions of a circular tapered groove centered about aperture 1516, thereby allowing glenoid component 1520 to be coupled to baseplate 1510 in any of a variety of relative orientations.

Glenoid component 1520, baseplate 1510 and central anchor screw 918 may each comprise a metal, or other suitable biocompatible material, e.g., titanium (Ti) and/or cobalt alloys (e.g., cobalt-chromium (CoCr), cobalt-chromium-molybdenum (CoCrMo)).

The two-piece baseplate assembly 1510, comprising metal baseplate 1510 and central anchor screw 918 coupled to one another utilizing an implant locking screw are universal and, so, may also be utilized in a reverse glenosphere system 1700 as shown in FIG. 17. For example, once central anchor screw 918 is secured within patient bone and baseplate 1510 is secured to central anchor screw 918, rather than press-fitting glenoid component 1520 into secured baseplate 1510, dual-taper adapter (trunnion) 1730 may be utilized to couple glenosphere 1220 to baseplate 1510.

In some such embodiments, dual-taper adapter 1730 comprises a central portion 1736, a first tapered portion 1732 extending away from central portion 1736 a first direction, and a second tapered portion 1734 extending away from central portion 1736 a second direction opposite the first direction. First tapered portion 1732 is configured to taper or friction-fit within recess 1224 in the underside of glenosphere 1220. Second tapered portion 1734 is configured to taper or friction-fit within the top portion of aperture 1516 in the topside of baseplate 1510.

In some embodiments, dual-taper trunnion 1730 may be secured to baseplate 1510 utilizing a similar implant locking screw disposed into a central aperture of dual-taper trunnion. Likewise, glenosphere 1220 may be secured to dual-taper trunnion 1730 utilizing another similar implant locking screw disposed through aperture 1229 and engaging with complementary threads in at least one of dual-taper trunnion 1730 and/or the head of the implant locking screw securing dual-taper trunnion 1730 to baseplate 1510. In yet other embodiments, an implant locking screw may be eliminated by utilizing a single implant locking screw having sufficient length to seat within aperture 1229, pass through glenosphere 1220 and the central aperture of dual-taper trunnion 1730, and engage its threads with complementary threads in at least one of baseplate 1510 (e.g., in an inner wall of the top portion of aperture 1516) and/or the head of implant locking screw 927 (see, e.g., FIGS. 16A-16E) securing baseplate 1510 to central anchor screw 918 below.

FIGS. 18A-19B illustrate example embodiments similar to those illustrated in FIGS. 9-11, however, substituting a first type of glenoid wedge component 1820 for glenoid component 920 in FIGS. 18A and 18B and substituting a second type of glenoid wedge component 1920 for glenoid component 920 in FIGS. 19A and 19B. Accordingly, all features in FIGS. 18A-18B and 19A-19B have the same numerals as corresponding features described in connection with FIGS. 9-11, except differences specifically described below for first and/or second glenoid wedge components 1820, 1920.

As illustrated in FIGS. 18A and 18B, glenoid wedge component 1820 comprises arcuate top surface 122 having an aperture (not shown but see, e.g., 929 in FIG. 9) disposed therein. A first portion 1821a, e.g., half, of the underside of glenoid wedge component 1820 extends in a first plane, and a second portion 1821b e.g., half, of the underside of glenoid wedge component 1820 extends from first portion 1821a in a second plane that rotated or offset by a predetermined angle compared to the first plane. In some embodiments, the first plane is substantially perpendicular (i.e., normal) to an axial direction of extension of central anchor screw 918. In embodiments as shown in FIG. 18A, the predetermined angle of the second plane with respect to this first plane is approximately 5°. In embodiments as shown in FIG. 18B, the predetermined angle of the second plane with respect to this first plane is approximately 7°. However, this disclosure is not so limited and any other suitable angle(s) is/are also contemplated. Disk-like component 924 is molded, overmolded, or press-fit into a center of the underside of glenoid wedge component 1820, as illustrated and as previously described.

As illustrated in FIGS. 19A and 19B, glenoid wedge component 1920 comprises arcuate top surface 122 having an aperture (not shown but see, e.g., 929 in FIG. 9) disposed therein. An underside 1921 of glenoid wedge component 1920 extends in a plane that rotated or offset by a predetermined angle compared to a plane perpendicular (i.e., normal) to an axial direction of extension of central anchor screw 918. In embodiments shown in FIG. 19A, the predetermined angle is approximately 5°. In embodiments shown in FIG. 19B, the predetermined angle is approximately 7°. However, this disclosure is not so limited and any other suitable angle(s) is/are also contemplated. Disk-like component 924 is likewise molded, overmolded, or press-fit into a center of underside 1921 of glenoid wedge component 1920, as illustrated and as previously described.

Moreover, while the glenoid wedge components 1820, 1920 shown in FIGS. 18A-19B are utilized in conjunction with, e.g., the systems of FIGS. 9-11, conversion of at least an underside portion of any glenoid baseplate or, where such baseplates are not utilized, glenoid component, to have similar planar features are also contemplated with any systems herein.

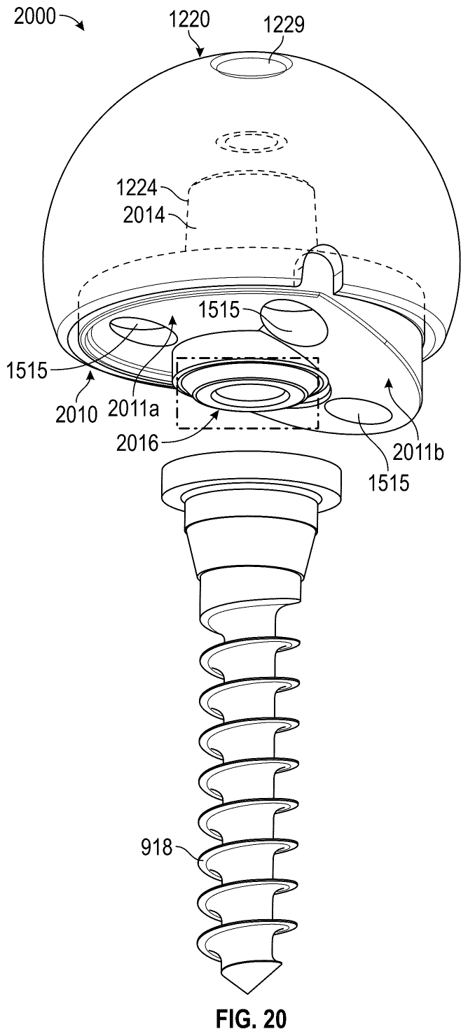

FIG. 20 illustrates another example embodiment of a modular system 2000 for shoulder arthroplasty. System 2000 comprises a baseplate 2010, central anchor screw 918, and glenosphere component 1220 comprising recess 1224 and aperture 1229 for receiving an implant locking screw. In some embodiments, system 2000 may be configured for onsetting substantially on a surface of sub-chondral bone.

Baseplate 2010 comprises at least a two-piece assembly of the baseplate 2010 itself and central anchor screw 918. Similar to baseplate 1210 of FIG. 12, baseplate 2010 comprises a plurality of peripheral apertures 2015 for receiving respective peripheral screws (not shown but see 312 in FIG. 3), a central tapered portion 2014 extending away from a top surface of baseplate 2010 (and configured to taper or friction-fit into recess 1224 in the underside of glenosphere 1220), and at least the bottom portion of metal disk-like component 924 disposed and/or machined into an underside thereof.

Central tapered portion 2014 comprises a recess and aperture 2016 in its top surface. At least a central portion of aperture 2016 extends entirely through baseplate 2010 such that an implant locking screw (not shown but see, e.g., 927 in FIG. 9) is received through recess 2016, extends out through aperture 2016 in the underside of baseplate 2010, and its thread engage with complementary threads of one or both of metal disk-like component 924 and the head of central anchor screw 918.

Baseplate 2010 also comprises a plurality of peripheral apertures 2015 each receiving a peripheral screw (not shown but see, e.g., 312 in FIGS. 3 and 4) configured to secure baseplate 2010 to bone of the patient peripheral to central anchor screw 918.

An underside of baseplate 2010 may have features similar to the underside(s) of glenoid wedge components 1820, 1920 of FIGS. 18 and 19. For example, a first portion 2021a, e.g., half, of the underside of glenoid wedge component 2020 extends in a first plane, and a second portion 2021b e.g., half, of the underside of glenoid wedge component 2020 extends from first portion 2021a in a second plane that rotated or offset by a predetermined angle compared to the first plane. In some embodiments, the first plane is substantially perpendicular (i.e., normal) to an axial direction of extension of central anchor screw 918. In embodiments, the predetermined angle of the second plane with respect to this first plane is approximately 5°, approximately 7°, or any other suitable angle.

Baseplate 2010 comprises a metal, or other suitable biocompatible material, e.g., titanium (Ti) and/or cobalt alloys (e.g., cobalt-chromium (CoCr), cobalt-chromium-molybdenum (CoCrMo)).

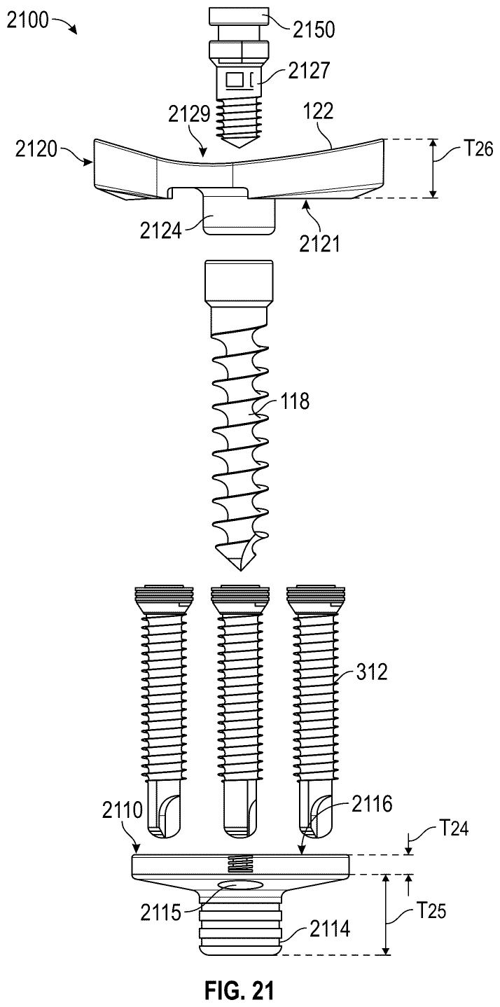

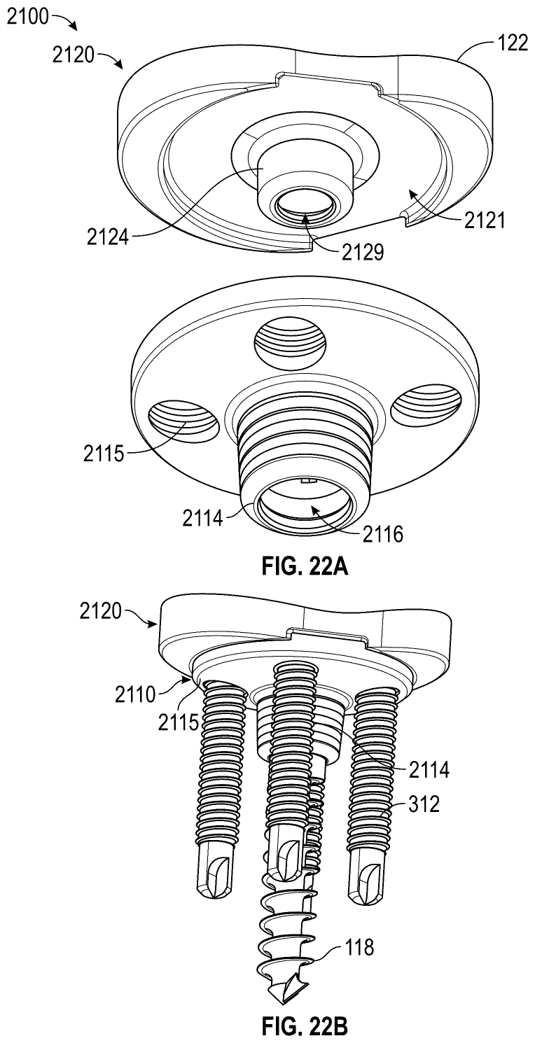

FIGS. 21, 22A and 22B illustrate another example all-metal embodiment of a modular system 2100 for shoulder arthroplasty. System 2100 comprises a baseplate 2110 and a glenoid component 2120. In some such embodiments, baseplate 2110 may be configured for insetting below sub-chondral bone.

Baseplate 2110 comprises a two-piece assembly including the baseplate 2110 itself and central compression screw 118, configured to properly align, secure and compress baseplate 2110 against and/or within patient bone as previously described. Baseplate 2110 comprises a central bossed portion 2114 extending from its underside and having similar features to those of baseplate 310 of FIG. 3, for example, comprising a recess and aperture 2116 in its top surface. At least a central portion of aperture 2116 extends entirely through baseplate 2110 such that central compression screw 118 is received through recess 2116, extends out through aperture 2116 in the underside of baseplate 2110, and a head of central compression screw 118 is entirely disposed within recess 2116, below a substantially flat upper surface of baseplate 2110 once central compression screw 118 is properly tightened into the bone of the patient. In some embodiments, aperture 2116 has sufficient diameter to accommodate central compression screws having diameters of 6.5 mm and/or 8.0 mm.

In some embodiments, an outer surface of at least a portion of central bossed portion 2114 comprises a porous metallic layer as previously described. Baseplate 2110 also comprises a plurality of peripheral apertures 2115 each receiving a respective peripheral screw 312 as previously described.

As illustrated in FIG. 21, a top surface of baseplate 2110 has a thickness T24 of 2.0-3.0 mm, inclusive, for example 2.08 mm. And a base of central bossed portion 2114 may have a diameter of approximately 11.5 mm while a distal end of central bossed portion 2114 may have a diameter of approximately 10.5 mm, which may be approximately 1.5 mm larger than central bossed portions of some prior described embodiments of this disclosure. As illustrated in FIG. 21, central boss 2114 extends a length T25 away from the underside of baseplate 2110. Example values for T25 include 10.5-11.5 mm, inclusive, for example, 10.83 mm. However, this disclosure is not so limited and T24 and T25 may have any suitable values.

Glenoid component 2120 comprises arcuate top surface 122 as previously described. Glenoid component 2120 comprises a central tapered portion 2124 extending from its underside and configured to press and taper or friction fit within aperture 2116 of baseplate 2110 such that the underside of glenoid component 2120 is in direct contact with the upper surface of baseplate 2110. Glenoid component 2120 has a thickness T26 measured between a topmost peripheral point of arcuate surface 122 and a corresponding bottom-most peripheral point of the underside of glenoid component 2120. Example values for T26 include 7.0-8.0 mm, inclusive, for example, 7.4 mm.

In some embodiments, at least a portion 2121 of the underside of glenoid component 2120 may be recessed such that at least a portion of baseplate 2110 may sit therein when glenoid component 2120 and baseplate 2110 are properly situated with respect to one another.

Glenoid component 2120 also comprises an aperture 2129 disposed therethrough configured to receive an implant locking screw 2127 that secures glenoid component 2120 to baseplate 2110 by engaging its threads with complementary threads in at least one of baseplate 2110 (e.g., in an inner wall of the top portion of aperture 2116) and/or the head of central compression screw 118. And, in some embodiments, a screw cap cover 2150 is configured to fit within aperture 2129 in arcuate top surface 122 of glenoid component 2120 and over implant locking screw 2127 to, thereby, ensure a substantially continuous and smooth transition between immediately adjacent edges of arcuate top surface 122 and screw cap cover 2150.

Glenoid component 2120 has a thickness T26 as measured between a topmost peripheral point of arcuate surface 122 and a corresponding bottom-most peripheral point of the underside of glenoid component 2120. In some embodiments, system 2100 is dimensioned such that a total thickness of system 100 extending above patient bone is approximately 4.2 mm.

Glenoid component 2120 and baseplate 2110 may each comprise a metal, or other suitable biocompatible material, e.g., titanium (Ti) and/or cobalt alloys (e.g., cobalt-chromium (CoCr), cobalt-chromium-molybdenum (CoCrMo)).

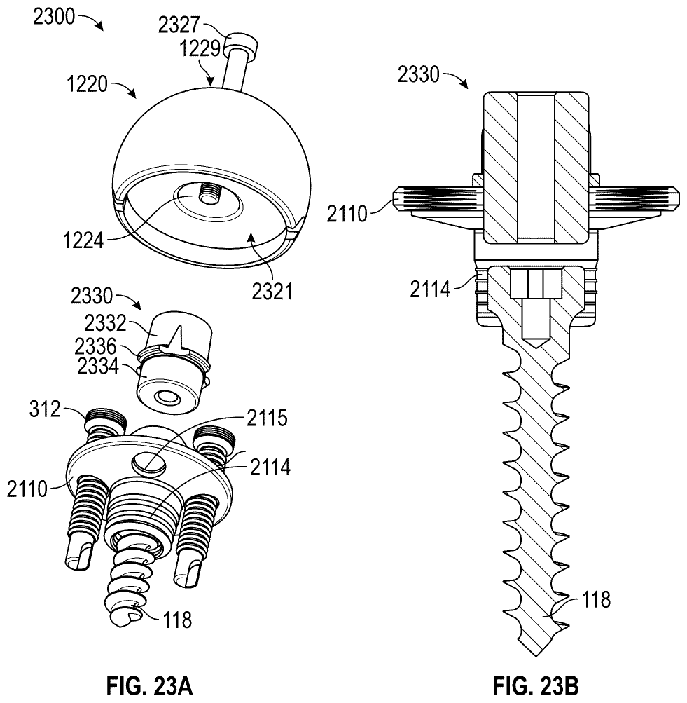

The two-piece baseplate assembly 2110, comprising metal baseplate 2110 and central compression screw 118, are universal and, so, may also be utilized in a reverse glenosphere system 2300 as shown in FIGS. 23A and 23B. For example, baseplate 2110 is secured against and/or within patient bone (e.g., central compression screw 118 and peripheral screws 312 are each properly secured), rather than press-fitting and then securing glenoid component 2120 into secured baseplate 2110, a dual-taper adapter (trunnion) 2330 may be utilized to couple glenosphere 1220 to baseplate 2110.

In some embodiments, dual-taper trunnion 2300 may be substantially similar to dual-taper trunnion 1730 of FIG. 17, having a similar central portion 2336, first tapered portion 2332, and second tapered portion 2334.

Implant locking screw 2327 may be disposed through aperture 1229 extend through glenosphere 2320, through a central aperture in dual-taper adapter 2330 and enmesh threads at its distal tip with complementary threads in a top of central compression screw 118, thereby securing both glenosphere 2320 and dual-taper adapter 2330 to baseplate 2110 utilizing the single implant locking screw. This arrangement may be more easily appreciated in the cutaway view of at least adapter 2330, baseplate 2110 and central compression screw 118 shown in FIG. 23B. In this way, implant locking screw 2327 eliminates a need for a separate implant locking screw for dual-taper adapter 2330 and, so, reduces unnecessary modularity and the attendant risk for practitioner confusion. In some embodiments, a head of implant locking screw 2327 comprises tightening features compatible with a hex drive. However, this disclosure is not so limited and the head may comprise tightening features compatible with any other suitable type of drive, for example Torx drive.

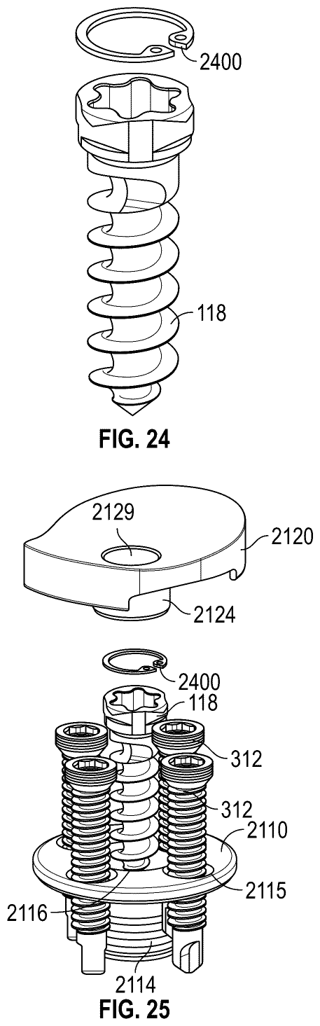

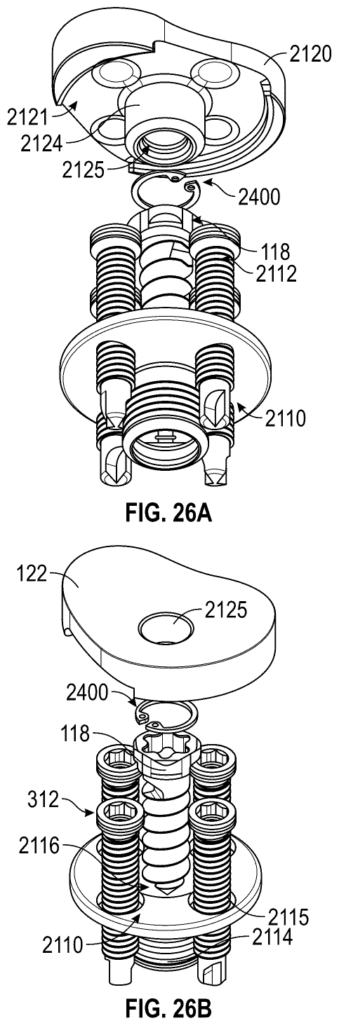

In some embodiments, for example as illustrated in FIGS. 24-26, baseplate 2110 and central compression screw 118 may be pre-assembled and retained, utilizing a screw snap ring 2400, before implant packaging, for example by the manufacturer. FIG. 25 illustrates an exploded view of how central compression screw 118 is disposed through aperture 2116 in baseplate 2110 and retained by pressing screw snap ring 2400 into aperture 2116 and over central compression screw 118. FIG. 25 also illustrates how peripheral screws 312 are disposed through apertures 2115 and extend alongside central bossed portion 2114, and how glenoid component 2120 is fit into baseplate 2110 such that central tapered portion 2124 fits into aperture 2116 and such that aperture 2129, which passes centrally through glenoid component 2120 and central tapered portion 2124, aligns with central compression screw 118. FIG. 26A illustrates a bottom perspective view of system 2100 and FIG. 26B illustrates a top perspective view of system 2100.

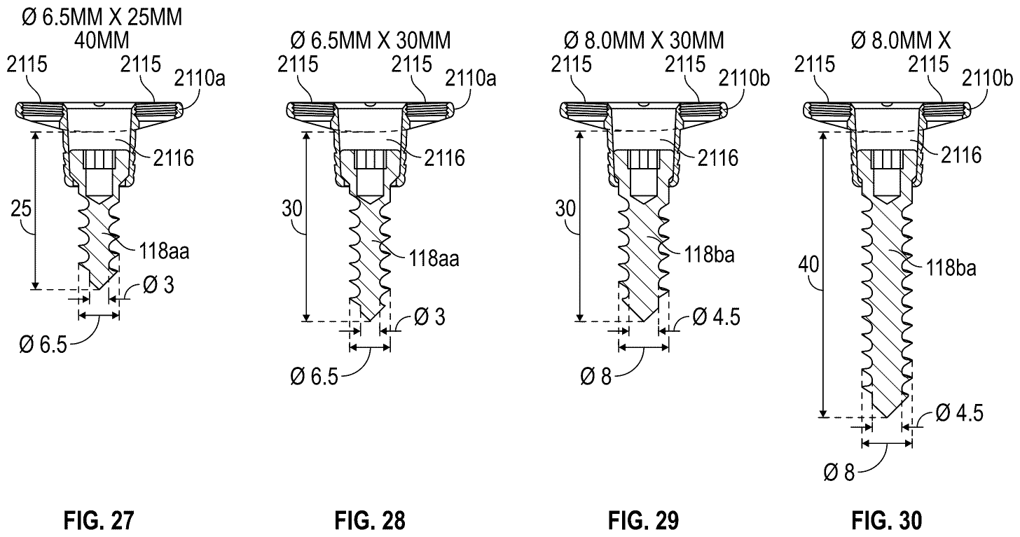

FIGS. 27-30 illustrate different implementations of baseplate 2110 accommodating different implementations of central compression screw 118 that have different dimensions. For example, in FIG. 27, central compression screw 118aa has a length of approximately 25 mm, a shaft diameter of approximately 3 mm, and teeth diameter of approximately 6.5 mm. In FIG. 28, central compression screw 118ab has a length of approximately 30 mm but the same shaft diameter and teeth diameter as in FIG. 27. In FIG. 29, central compression screw 118ba has a length of approximately 30 mm, a shaft diameter of approximately 4.5 mm, and teeth diameter of approximately 8 mm. In FIG. 30, central compression screw 118bb has a length of approximately 40 mm but the same shaft diameter and teeth diameter as in FIG. 29. One aspect illustrated between FIGS. 27-30 is that a head of the central compression screws 118aa, 118ab having teeth diameters of 6.5 mm may be enlarged from original to match or have substantially the same dimensions as the heads of the central compression screws 118ba, 118bb having teeth diameters of 8 mm. In such embodiments, a same baseplate may be utilized for applications using either 6.5 mm or 8 mm central compression screws 118 that have the same head dimensions.

Contrarily, in some other embodiments where central compression screws 118 with different diameter teeth have different dimensioned heads, separate implementations of baseplate 2110 may be utilized, each having a differing sized central bossed portion 2114 to accommodate the heads of central compression screw 118 having a particular size.

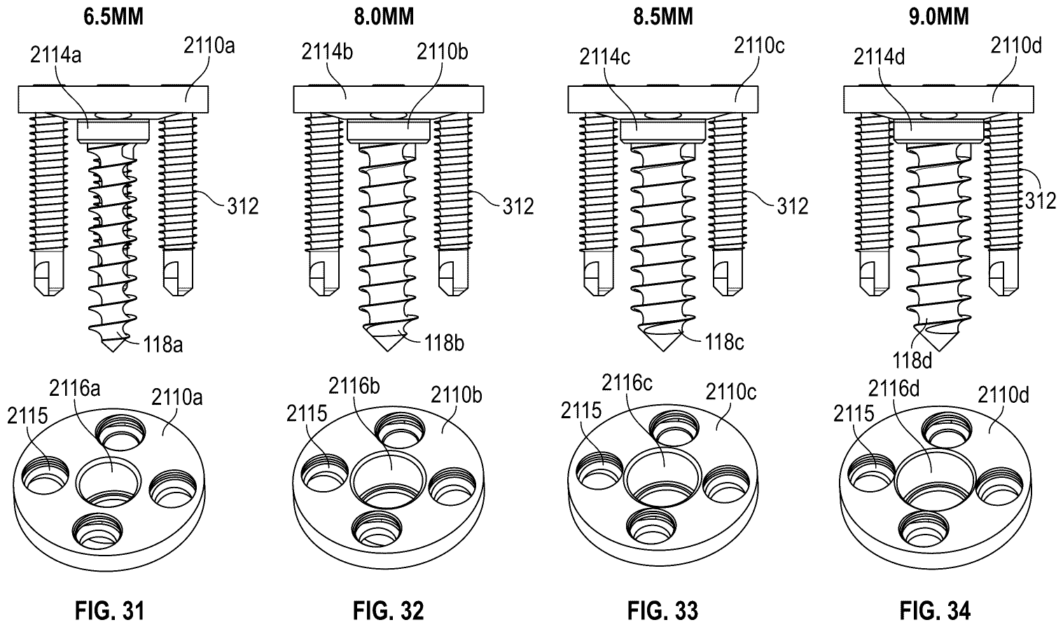

FIGS. 31-34 illustrate embodiments of baseplate 2110 having central bossed portions with different diameters, e.g., baseplates 2110a-2110d, to accommodate different-dimensioned central compression screws 118, e.g., example central compression screws 118a-118d. In some such embodiments, central bossed portions 2114a-2114d of baseplates 2110a-2110d may also have different depths (or lengths) of extension compared to prior-described central bossed portions and/or from one another, for example, having a shorter length of extension in some cases and/or a different diameter according to and/or accommodating the size of a compatible central compression screw 118a-d.

For example, in FIG. 31, central compression screw 118a has teeth diameters of approximately 6.5 mm and aperture 2116a of baseplate 2110a is, accordingly, sized appropriately. In FIG. 32, central compression screw 118b has teeth diameters of approximately 8 mm and aperture 2116b of baseplate 2110b is, accordingly, sized appropriately, e.g., larger than aperture 2116a. In FIG. 33, central compression screw 118c has teeth diameters of approximately 8.5 mm and aperture 2116c of baseplate 2110c is, accordingly, sized appropriately, e.g., larger than apertures 2116a, 2116b. In FIG. 34, central compression screw 118d has teeth diameters of approximately 9 mm and aperture 2116d of baseplate 2110d is, accordingly, sized appropriately, e.g., larger than apertures 2116a-2116c. Some such embodiments provide a larger diameter central compression screw to address increased patient bone loss and/or poor patient bone quality.

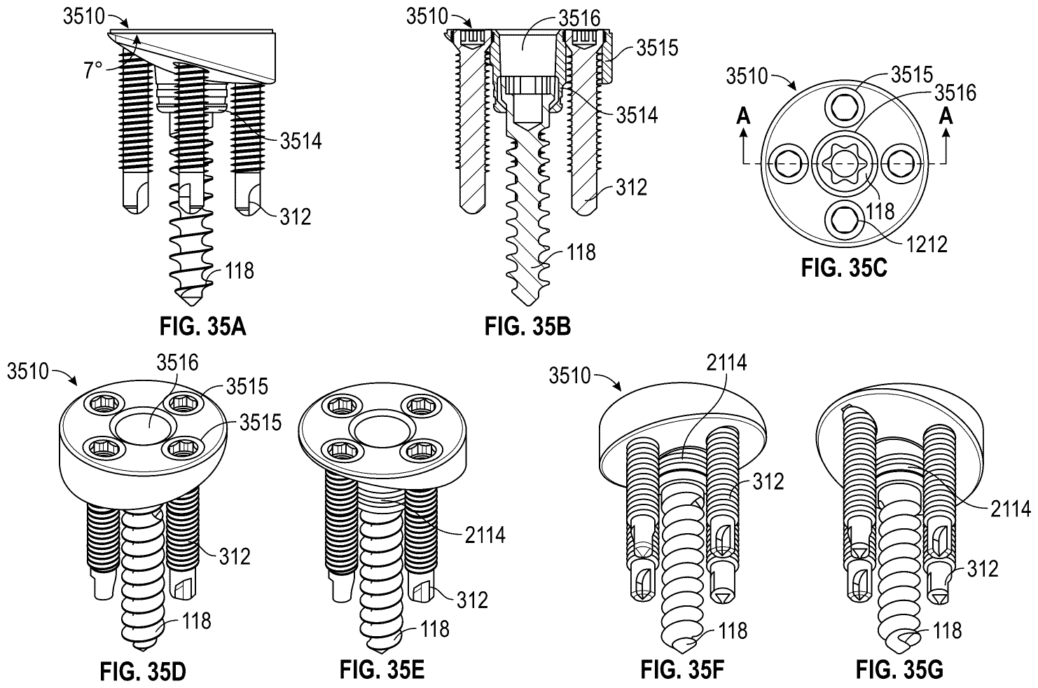

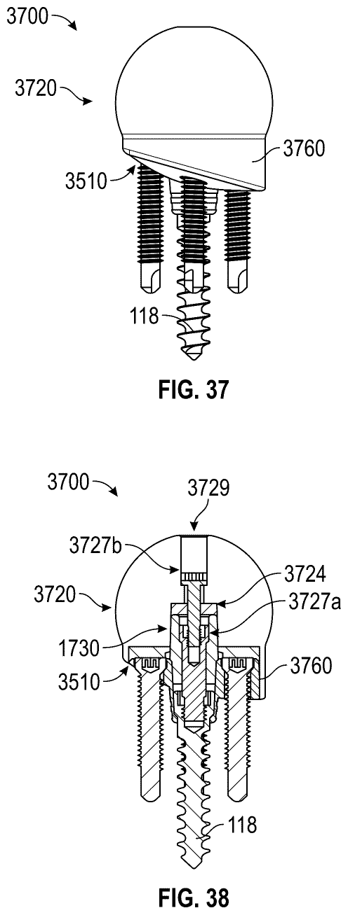

FIGS. 35A-35G illustrate different views of an alternative baseplate wedge 3500 for utilization with, for example, the other components of system 2100 or other components of system 3700 of FIG. 37, as will be described below. FIG. 35A illustrates a side view. FIG. 35B illustrates a cutaway view. FIG. 35C illustrates a top view. FIG. 35D illustrates a top perspective view. FIG. 35E illustrates another top perspective view. FIG. illustrates a bottom perspective view. FIG. 35G illustrates another bottom perspective view.

Baseplate wedge 3520 comprises a substantially planar top surface that is perpendicular (i.e., normal) to an axial direction of extension of central compression screw 118. The top surface comprises an aperture 3516 and peripheral apertures 3515 substantially similar to corresponding apertures previously described in connection with baseplate 2100 of FIG. 21. An underside of baseplate wedge 3510 extends in a plane that is rotated or offset by a predetermined angle compared to the plane of the top surface. In some embodiments, the predetermined angle is approximately 7°. However, this disclosure is not so limited and any other suitable angle(s) is/are also contemplated. Such 7 mm full wedge embodiments may correlate to the AltiVate Anatomic All-poly 7 mm Augmented Glenoid Insert component, which enables utilization of aspects of the Instrumentation from that Augmented Glenoid system.

Baseplate wedge 3510 also comprises a central bossed portion 3514 extending from its underside substantially similar to central bossed portion 2114 of baseplate 2110, except for those differences that result from the underside of baseplate wedge 3510 being rotated or offset compared to the plane of the top surface (e.g., the varying thickness of baseplate wedge 3510).

Baseplate wedge 3510 may comprise a metal, or other suitable biocompatible material, e.g., titanium (Ti) and/or cobalt alloys (e.g., cobalt-chromium (CoCr), cobalt-chromium-molybdenum (CoCrMo)).

In some embodiments as illustrated in FIGS. 37-39B, system 3700 includes glenosphere component 3720 configured to be secured to baseplate wedge 3510 previously described in connection with FIG. 35. Glenosphere component 3720 may be a version or embodiment of glenosphere component 1220. Glenosphere component 3720 comprises a skirt 3760 extending from the convex portion of glenosphere component 3720. In some embodiments, skirt 3760 has a substantially cylindrical form and extends from a top edge to a bottom edge. The top edge forms a border with the adjacent convex portion in a first plane that is substantially parallel to a top surface of baseplate 3510 when glenosphere component 3720 is properly secured thereto. The bottom edge substantially resides in a second plane that is rotated compared to the first plane by an angle substantially equal to the wedge angle of baseplate 3510 (e.g., 7°). In this way, skirt 3760 substantially surrounds and is generally in contact with or immediately adjacent a perimeter of the wedged portion of baseplate 3510 and, thereby, shares a load placed on baseplate 3510 in vivo.

The cutaway view of system 3700 in FIG. 38 illustrates, in more detail, how dual-taper adapter 1730 (see also, FIG. 17) and glenosphere component 3720 (and/or, e.g., glenosphere component 1220) couple to a corresponding baseplate 3510. In some embodiments, baseplate 3510 is secured to patient bone via by central compression screw 118, disposed within recess and/or aperture 3516 in the top surface of baseplate 3510. One tapered end of dual-taper adapter 1730 is then seated within aperture 3516, over the head of central compression screw 118. An implant locking screw 3727a is disposed through a central aperture in dual-taper adapter 1730 and its distal threads engage with mating threads in the head of central compression screw 118. Glenosphere component 3700 may then be seated onto the opposite tapered end of dual-taper adapter 1730 (e.g., the opposite tapered end being received within recess 3724). Skirt 3760 substantially surrounds the wedged portion of baseplate 3510 and, thereby, shares a load placed on baseplate 3510 in vivo. Another implant locking screw 3727b may be disposed into and seated within aperture 3729 such that its distal threads engage with mating threads in a head of implant locking screw 3727a.

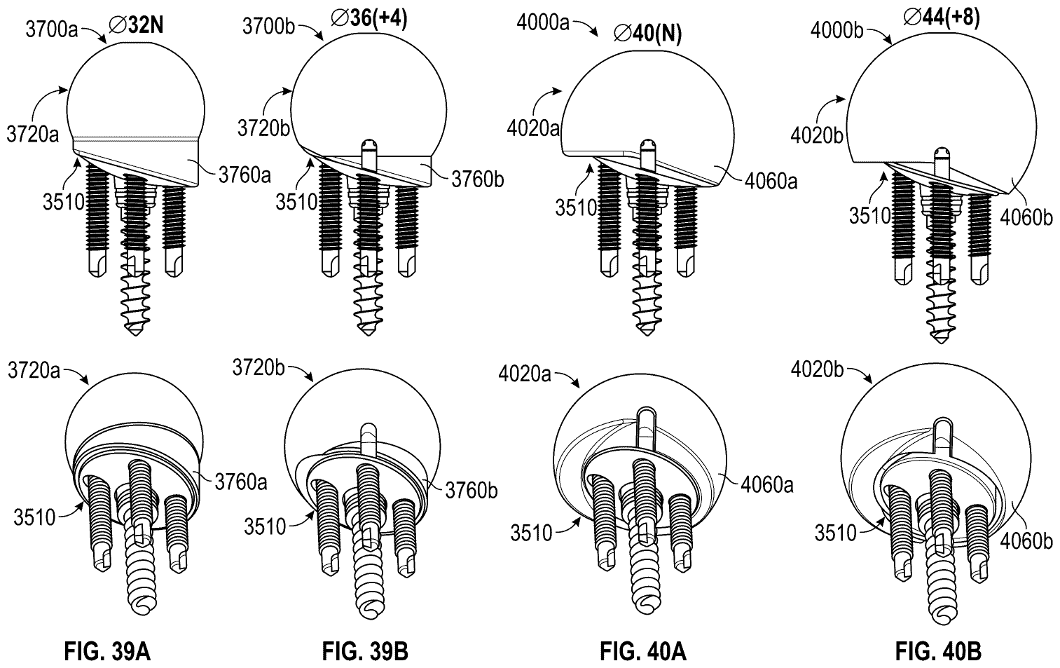

FIG. 39A shows different views of a version of system 3700, system 3700a, comprising baseplate 3510 and a glenosphere component 3700a, corresponding to the COR 32N embodiment shown in FIG. 36. FIG. 39B shows different views of another version of system 3700, system 3700b, comprising baseplate 3510 and a glenosphere component 3700b, corresponding to the COR 32+4 embodiment shown in FIG. 36.

Glenosphere component 3720, 3720a, 3720b may comprise a metal, or other suitable biocompatible material, e.g., titanium (Ti) and/or cobalt alloys (e.g., cobalt-chromium (CoCr), cobalt-chromium-molybdenum (CoCrMo)).

FIG. 40A shows different views of a system 4000a, similar to systems 3700a, 3700b, but comprising baseplate 3510 and a glenosphere component 4020a. FIG. 40B shows different views of another system 4000b, similar to systems 3700a, 3700b, but comprising baseplate 3510 and a glenosphere component 4020b.

Glenosphere components 4020a and 4020b may be substantially the same as glenospheres 3720a, 3720b except replacing skirts 3760a, 3760b with respective hoods 4060a, 4060b, which each extend the convex shape of respective glenosphere component 4020a, 4020b over an arc extending between where the top and bottom edges of the respective skirt 3760a, 3760b would be (see, e.g., FIGS. 39A and 39B. Accordingly, hoods 4060a, 4060b extend the convex surface by a radial angle substantially equal to the wedge angle of baseplate 3510 (e.g., 7°). In this way, hoods 4060a, 4060b substantially surround a perimeter of the wedged portion of baseplate 3510 and, thereby, share a load placed on baseplate 3510 in vivo. For purposes of example and not limitation, glenosphere component 4020a corresponds to the COR 44+8 embodiment shown in FIG. 36, while glenosphere component 4020b corresponds to the COR 40N embodiment shown in FIG. 36.

Glenosphere component 4020, 4020a, 4020b may comprise a metal, or other suitable biocompatible material, e.g., titanium (Ti) and/or cobalt alloys (e.g., cobalt-chromium (CoCr), cobalt-chromium-molybdenum (CoCrMo)).

FIGS. 41A and 41B illustrates several views of yet another modular system 4100 for shoulder arthroplasty, comprising a glenoid component 4120, a baseplate wedge 4110 having a full wedge, and a modular bossed central anchor screw 4118 which, itself, includes a proximal boss 4114 in place of baseplate 4100 having a central bossed portion, as previously described for several embodiments.

Baseplate wedge 4110 comprises a substantially planar top surface that is perpendicular (i.e., normal) to an axial direction of extension of central compression screw 118. The top surface comprises peripheral apertures 4115 substantially similar to corresponding apertures previously described in connection with baseplate 3510 of any of FIGS. 35A-35G. The top surface also comprises an aperture 4116 extending through baseplate wedge 4110. An underside of baseplate wedge 4110 extends in a plane that is rotated or offset by a predetermined angle compared to the plane of the top surface. In some embodiments, the predetermined angle is approximately 7°. However, this disclosure is not so limited and any other suitable angle(s) is/are also contemplated.

In contrast to some other embodiments, rather than baseplate wedge 4110 comprising a central bossed portion extending from its underside, bossed central anchor screw 4118 itself comprises a proximal boss 4114 that is configured to couple into aperture 4116 at the underside of baseplate 4110. For example, and not limitation, an inner surface of aperture 4116 may comprise a locking feature 4119a and proximal boss 4114 may comprise a complementary locking feature 4119b configured to engage with locking feature 4119a. In some embodiments, locking features 4119a and 4119b comprise a bayonet lock type mechanism. In some embodiments, proximal boss 4114 comprises threads 4115 configured to engage with patient bone as bossed central anchor screw 4118 is driven to a desired depth into the patient bone. However, proximal boss 4114 could additionally or alternatively comprise a porous layer configured to aid bone adhesion and ingrowth thereto. Accordingly, a practitioner may drive bossed central anchor screw 4118 to a desired depth into patient bone, such that threads 4115 (where present) bite into patient bone and secure bossed central anchor screw 4118 therein. The practitioner may then snap baseplate wedge 4110 onto bossed central anchor screw 4118 by inserting a proximal end of bossed central anchor screw 4118 into aperture 4116 from the underside of baseplate wedge 4110 until locking features 4119a and 4119b engage one another. In such embodiments, snapping baseplate 4110 onto bossed central anchor screw 4118 allows bossed central anchor screw 4118 to compress baseplate 4110 into the desired position once assembled, similar to central compression screw 118.

As illustrated in FIGS. 41A and 41B, glenoid component 4120 may be substantially as previously described in connection with, for example FIG. 26. Accordingly, glenoid component 4120 may comprise an arcuate top surface 122, a recessed portion 4121 in its underside, and a central tapered portion 4124 extending from an underside thereof, an aperture 4129 being disposed in top surface 122, extending through glenoid component 4120, and configured to receive an implant locking screw (not shown). Such an implant locking screw may comprise distal threads configured to engage with complementary threads in at least one of baseplate wedge 4110 (e.g., in an inner wall of the top portion of aperture 4116) and/or an inner surface of proximal bossed 4114 of bossed central anchor screw 4118.

Glenoid component 4120 and baseplate wedge 4110 may each comprise a metal, or other suitable biocompatible material, e.g., titanium (Ti) and/or cobalt alloys (e.g., cobalt-chromium (CoCr), cobalt-chromium-molybdenum (CoCrMo)).

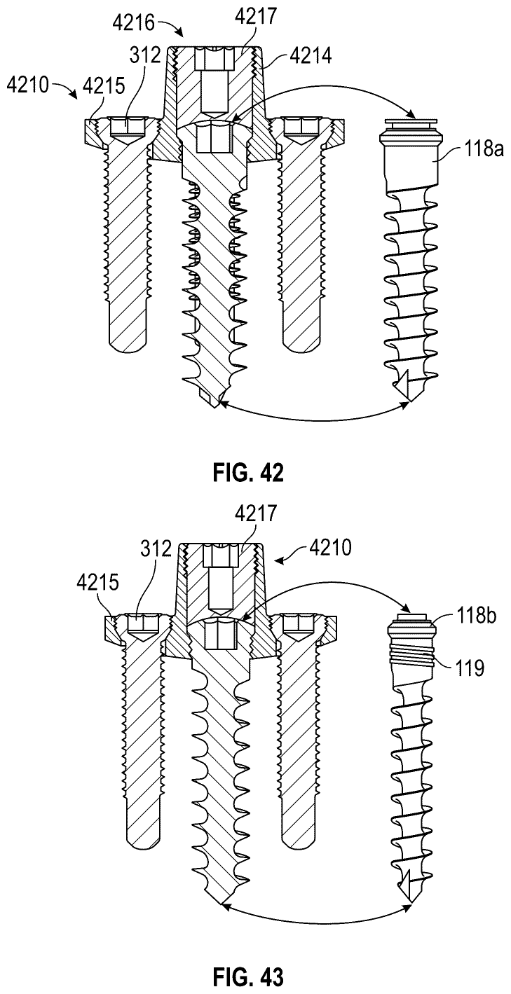

Throughout this disclosure a center screw is often utilized in a compressive role (e.g., central compression screw 118). That is, once properly disposed within patient bone and tightened, central compression screw 118 compresses a baseplate or anchor boss into position against and/or within patient bone. Any embodiments disclosed herein, where central compression screw 118 is utilized to secure another component (e.g., a baseplate or anchor boss) to patient bone, may have several interrelated features between central compression screw 118 (e.g., 118a in FIGS. 42 and 118b in FIG. 43) and the aperture through and in which it is disposed.

For example, and not limitation, FIG. 42 illustrates a cutaway view of an example baseplate 4210 and central compression screw 118a. Baseplate 4210 comprises a central bossed portion 4214 extending from its top side, an aperture 4216 disposed through central bossed portion 4214 and baseplate 4210, and peripheral apertures 4215 for receiving peripheral screws 312 as previously described. In some embodiments, baseplate 4210 may be utilized in reverse arthroplasty procedures in which central bossed portion 4214 is configured to engage with a glenosphere component, for example, as described anywhere in this disclosure.

Aperture 4216 is configured to receive central compression screw 118a therethrough such that a head portion of central compression screw 118a seats against the reduced diameter of a distal portion of aperture 4216. Aperture 4216 may comprise one or more sets of threads, for example an upper set configured to engage with complementary threads of a set screw 4217, or implant locking screw as previously described, and a lower set configured to engage with complementary threads 119 on a head of central compression screw 118b, where present (see, e.g., FIG. 43).

In embodiments according to FIG. 42, a head portion of central compression screw 118a has substantially smooth outer sides at least in that it does not comprise threads configured to engage with the lower set of complementary threads within the reduced-diameter distal portion of aperture 4216. Accordingly, central compression screw 118a does not threadingly engage with baseplate 4210 in FIG. 42. Set screw 4217 also comprises threads configured to threadingly engage with the upper set of complementary threads in the proximal portion of aperture 4216. When properly seated within aperture 4216, as illustrated, set screw 4217 may be configured to prevent central compression screw 118a from backing out in vivo. In some embodiments, threads of set screw 4217 may have an opposite “handedness” direction from those of central compression screw 118a (e.g., left if those of screw 118a are right, right if those of screw 118a are left) so as to resist and/or counter in vivo torque imparted on the assembly. This disclosure also contemplates, but does not require, utilizing this opposite handedness for threads of any implant locking screw described herein.

As a contrasting but not limiting example, FIG. 43 illustrates a cutaway view of baseplate 4210, set screw 4217 and central compression screw 118b having threads 4319 complementary to the lower threads within the reduced-diameter distal portion of aperture 4216 on its head portion. Accordingly, when properly seated within aperture 4216, central compression screw 118b threadingly engages with baseplate 4210.

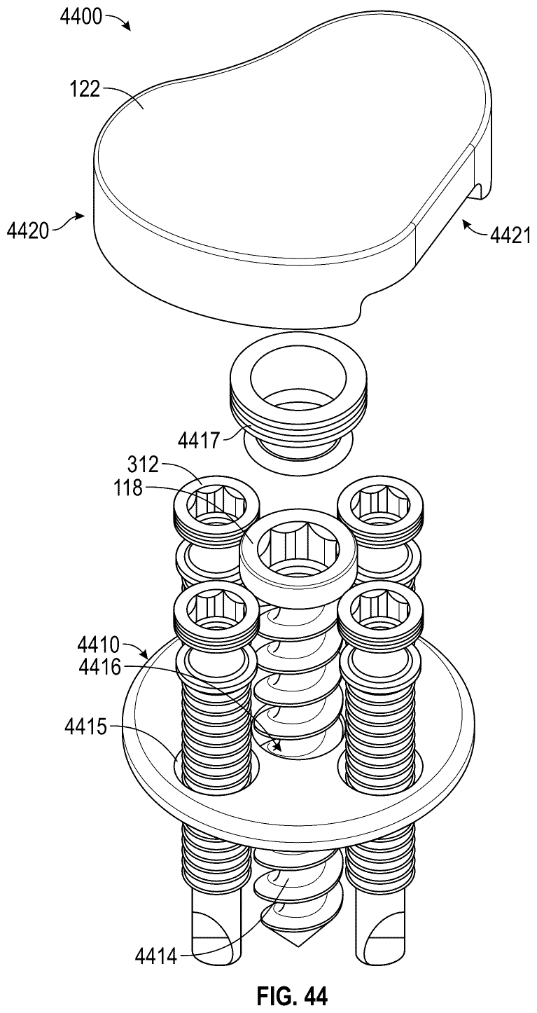

The use of a set screw to lock a center screw is not limited to embodiments shown in FIGS. 42 and 43. For example, FIG. 44 illustrates an embodiment of a modular system 4400 for use in shoulder arthroplasty. System 4400 comprises a glenoid component 4422 and a baseplate 4410 anchored to bone by central compression screw 118, which, itself, is locked in place by a set screw 4417.

Glenoid component 4420 may be substantially similar to previously described glenoid components (e.g., 2120 of FIGS. 21 and/or 4120 of FIG. 41), comprising arcuate top surface 122 and, in some cases, a recessed portion 4421 in its underside. While not visible in FIG. 44, glenoid component 4420 comprises a central tapered portion extending from the underside of glenoid component 4420 within recessed portion 4421 (see, e.g., 4124 in FIG. 41). While also not illustrated in FIG. 44, arcuate top surface 122 may comprise an aperture (see, e.g., 4129 in FIG. 41) configured to receive an implant locking screw having distal threads configured to engage with complementary threads in set screw 4417, in a head of central compression screw 118, and/or in a sidewall of a central aperture 4416 in the top surface of baseplate 4410, as will be described below, thereby, threadingly securing glenoid component 4420 to baseplate 4410.

Baseplate 4410 comprises central aperture 4416 configured to receive central compression screw 118 and then set screw 4417. Accordingly, aperture 4416, central compression screw 118 and set screw 4417 may substantially correspond to and have similar features to those described in connection with FIGS. 42 and/or 43, except that central bossed portion 4414 extends from the underside of baseplate 4410 rather than from a top surface as in FIGS. 42 and 43.

In some such embodiments, an inner portion of set screw 4417 may comprise a tapered recess configured to receive the central tapered portion extending from the underside of glenoid component 4420 within recessed portion 4421 of glenoid component 4420. In some other embodiments, set screw 4417 may be sufficiently low profile (e.g., has a sufficiently short height) that the central tapered portion extending from the underside of glenoid component 4420 within recessed portion 4421 is configured to fully seat within and taper and/or friction-fit with aperture 4416 in the top surface of baseplate 4410 itself.

Glenoid component 4420, baseplate 4410 and set screw 4417 may each comprise a metal, or other suitable biocompatible material, e.g., titanium (Ti) and/or cobalt alloys (e.g., cobalt-chromium (CoCr), cobalt-chromium-molybdenum (CoCrMo)).

FIG. 45 illustrates yet another example embodiment of a modular system 4500 for shoulder arthroplasty. System 4500 comprises glenosphere component 1220 as previously described, baseplate 4410 of FIG. 44, configured to be anchored to patient bone by central compression screw 118, and a threaded glenosphere adapter 4530.

Baseplate 4410 is configured to receive central compression screw 118 through central aperture 4416. Once central compression screw 118 is properly set and tightened, rather than utilizing set screw 4417, at least a threaded portion 4534 of glenosphere adapter 4530 is threaded into the upper set of threads in the proximal portion of aperture 4416 substantially as set screw 4417 was in FIG. 44. A tapered portion 4532 of glenosphere adapter 4530 is disposed adjacent threaded portion 4534 and configured to extend away from a top surface of baseplate 4410 when threaded portion 4534 is properly threaded into aperture 4416. Once tapered portion 4532 is properly seated within recess 1224 of glenosphere component 1220 (not shown but see, e.g., FIG. 12), an implant locking screw 4527 may be disposed within aperture 1229 of glenosphere component 1220, into or through a central aperture of adapter 4539. Distal threads of implant locking screw 4527 are configured to engage with complementary threads within at least one of the central aperture of adapter 4539, aperture 4416 of baseplate 4410, or a head of central compression screw 118 as previously described.

Accordingly, glenosphere adapter 4530, as an integral component, is configured to both act as a set screw for central compression screw 118 and as the adapter for coupling glenosphere component 1220 to baseplate 4410. And the threading required for acting as a set screw also functions to anchor adapter 4530 to baseplate 4410. Adapter 4530 also improves depth accuracy by virtue of fewer components, each contributing an error margin to the depth accuracy stack. The above are all examples of adapter 4530, having one function, being simultaneously utilized for another, previously unrelated, function.

FIG. 46 illustrates yet another example embodiment of a modular system 4600 for shoulder arthroplasty. In some embodiments, system 4600 may be configured for onsetting substantially on a surface of sub-chondral bone. System 4600 does not comprise a baseplate. Rather, system 4600 comprises a bossed central anchor screw 4618 having a bossed head 4614 and threads configured to bite into the patient bone. In some embodiments, at least a portion of bossed head 4614 of bossed central anchor screw 4618 is coated or otherwise formed to have a porous metal coating configured to aid in bone adhesion thereto after implantation. In some embodiments, bossed head 4614 comprises a mating or locking feature (not shown but, e.g., teeth, protrusions and/or a ridge) configured to engage with a metal disk-like component 4624 of glenoid component 4620, as will be described in more detail below.

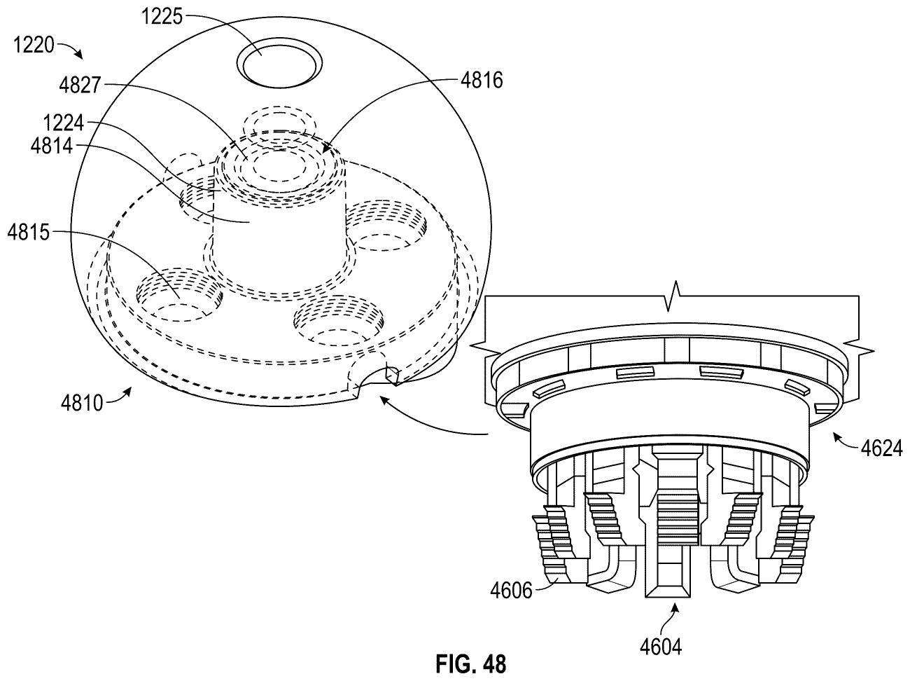

In some embodiments, glenoid component 4620 is a multi-piece assembly, substantially similar to glenoid component 920 of FIGS. 9-11 (e.g., including similar rough protrusions 4626) except, instead of utilizing metal disk-like component 924, a metal disk-like component 4624 is molded, integrally formed, cast, milled from a same piece of metal or metal alloy as, or inserted into a bottom surface of, glenoid component 4620. Metal disk-like component 4624 provides a key interface between glenoid component 4620 and bossed central anchor screw 4618. Metal disk-like component 4624 is substantially circular in form factor and comprises an aperture 4604 configured to receive an implant locking screw 4627 therethrough (implant locking screw 4627 is inserted into an aperture in arcuate top surface 122 not visible in FIG. 46). Metal disk-like component 4624 also comprises a plurality of deflectable fingers or extensions 4606 extending away from a bottom side thereof, together defining at least a distal portion of aperture 4604. When implant locking screw 4627 is disposed through aperture 4604, implant locking screw 4627 physically contacts and exerts a force against an inner surface of extensions 4606, causing them to deflect outwardly (e.g., in a radial direction) and mechanically engage with the mating or locking feature in the proximal boss 4614 of bossed central anchor screw 4618. As best illustrated in FIG. 48, a distal tip of each of extensions 4606 may comprise a notched, ridged, increased thickness, and/or respectively portioned discontinuous threadings configured to “latch” around or substantially immovably against a mating feature to which extensions 4606 couple glenoid component 4620.

Glenoid component 4620 and bossed central anchor screw 4614 may each comprise a metal, or other suitable biocompatible material, e.g., titanium (Ti) and/or cobalt alloys (e.g., cobalt-chromium (CoCr), cobalt-chromium-molybdenum (CoCrMo)).

FIG. 47 illustrates yet another example embodiment of a modular system 4700 for shoulder arthroplasty. System 4700 comprises glenoid component 4600 from FIG. 46 and anchor boss 514 and central compression screw 118 from FIG. 5. In some embodiments, system 4700 may be configured for onsetting substantially on, rather than below, a surface of sub-chondral bone.

Like embodiments shown in FIGS. 5 and 6, system 4700 also does not comprise a baseplate. Rather system 4700 comprises a first, two-piece assembly comprising anchor boss 514 and central compression screw 118. As previously described, aperture 516 is configured to receive central compression screw 118 such that a head of central compression screw 118 is entirely disposed within aperture 516 and below a substantially flat upper surface of anchor boss 514 when anchor boss 514 is properly placed in the prepared patient bone and secured therein by tightening central compression screw 118 to the desired degree. In some embodiments, similar to the proximal boss 4614 of bossed central anchor screw 4618 in FIG. 46, an inside surface of a proximal portion of aperture may comprise a mating or locking feature (not shown but, e.g., teeth, protrusions and/or a ridge) configured to engage with metal disk-like component 4624 of glenoid component 4620.

Metal disk-like component 4624 provides a key interface between glenoid component 4620 and at least one of an inner surface of aperture 516 within anchor boss 514 and an inside surface of aperture 516 center screw 118. When implant locking screw 4627 is disposed through aperture 4604, implant locking screw 4627 physically contacts an inner surface of extensions 4606 and causes them to deflect outwardly (e.g., in a radial direction), mechanically engaging with at least the above-described portion of anchor boss 514, within aperture 516. In some embodiments, at least distal threads (not shown) of implant locking screw 4627 may also be configured to threadingly engage with mating threads (not shown) in a head of central compression screw 118.

The bossed central anchor screw 4618 of FIG. 46 or the assembly of anchor boss 514 and compression screw 118 of FIGS. 5 and 6, but also shown in FIG. 47, may also be utilized as a part of a system 4800 comprising glenosphere 1220, as previously described, and a glenosphere baseplate 4810. Baseplate 4800 may have a substantially similar construction to baseplate 1210 from FIG. 12 or baseplate 2010 from FIG. 20 except, instead of utilizing metal disk-like component 924, metal disk-like component 4624 is molded, integrally formed, cast, milled from a same piece of metal or metal alloy as, or inserted into a bottom surface of, glenoid baseplate 4810.

Accordingly, aperture 4816 may extend to and become aperture 4604, with deflectable extensions 4606 disposed therearound, as shown in FIG. 48. Metal disk-like component 4624 provides a key interface between baseplate 4810 and bossed center anchor screw 4618 or anchor boss 514. When implant locking screw 4827 is disposed through apertures 4816 and 4604, implant locking screw 4627 physically contacts and exerts a force against an inner surface of extensions 4606, causing them to deflect outwardly (e.g., in a radial direction) and mechanically engage with the above-described portion of proximal boss 4614 of bossed central anchor screw 4618 or of aperture 516 of anchor boss 514. In some embodiments, at least distal threads (not shown) of implant locking screw 4627 may also be configured to threadingly engage with mating threads (not shown) in proximal boss 4614 or in a head of central compression screw 118 or of anchor boss 514.

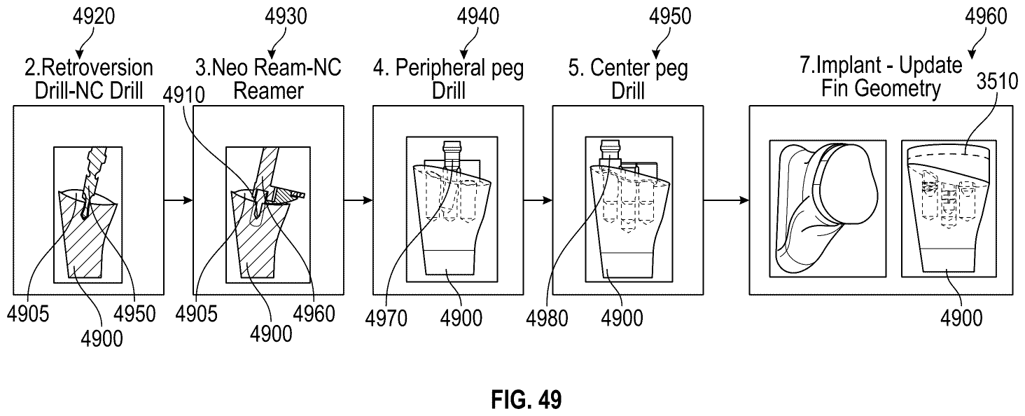

FIG. 49 illustrates a surgical technique overview for preparing a surface 4905 of patient humeral bone 4900 for using a convertible modular system for shoulder arthroplasty as described anywhere in this disclosure, according to some embodiments. For example only, baseplate 3510 previously described in connection with FIGS. 35A-35G is illustrated in the technique of FIG. 49. While certain steps are described in a particular order, the present disclosure is not so limited and a method or technique for preparing a surface of patient humeral bone may include fewer, additional, or alternative steps in the same or in any other suitable order.

In frame 4920, a retroversion drill is utilized to prepare a central hole into which a guide rod 4910 is secured. In frame 4930, a reamer 4960 is disposed on guide rod 4910 and surface 4905 of patient bone 4900 is reamed according to the requirements of the particular procedure. In frame 4940, where a baseplate comprises peripheral pegs or accommodates peripheral screws, a peripheral peg drill 4970 is utilized to drill holes that will ultimately accommodate any peripheral pegs disposed in a bottom surface of baseplate 3510 (see, e.g., 526 in FIG. 5) or peripheral screws 312 (see, e.g., FIGS. 35A-35G) as called for by the particular features of the baseplate. In frame 4950 a center peg drill is utilized to drill a hole that will ultimately accommodate portions of central bossed portion 3514 and/or central compression screw 118 (see, e.g., FIGS. 35A-35G). In frame 4960 baseplate 3510 is secured to the prepared surface 4905 of bone 4900 by, for example, properly inserting, driving and tightening central compression screw 118 and then, where utilized, peripheral screws 312 (see, e.g., FIGS. 35A-35G).

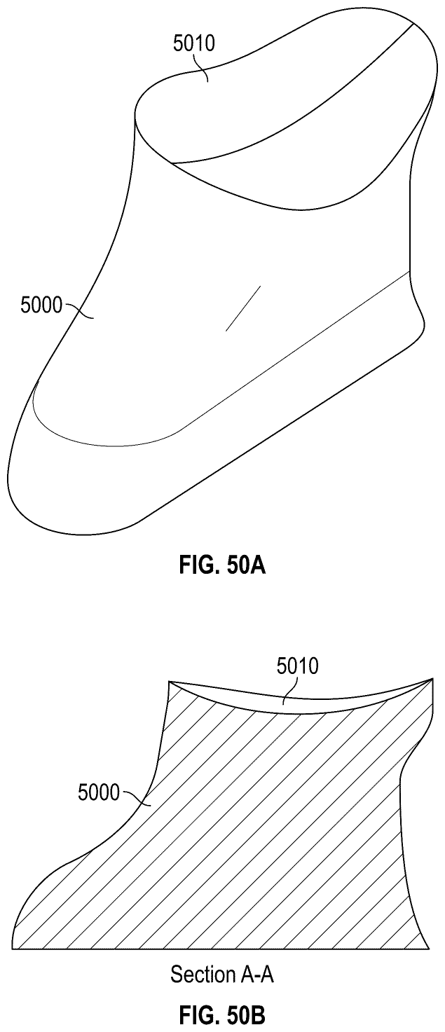

FIGS. 50A-53B illustrate a surgical technique overview for preparing patient scapular bone for using a convertible modular system for shoulder arthroplasty as described anywhere in this disclosure, according to some embodiments. FIG. 50A illustrates a perspective view of a portion of glenoid bone 5000 of a patient and illustrates at least a portion of a glenoid fossa 5010 (e.g., surface) thereof. FIG. 50A illustrates a cutaway view of the portion shown in FIG. 50A.

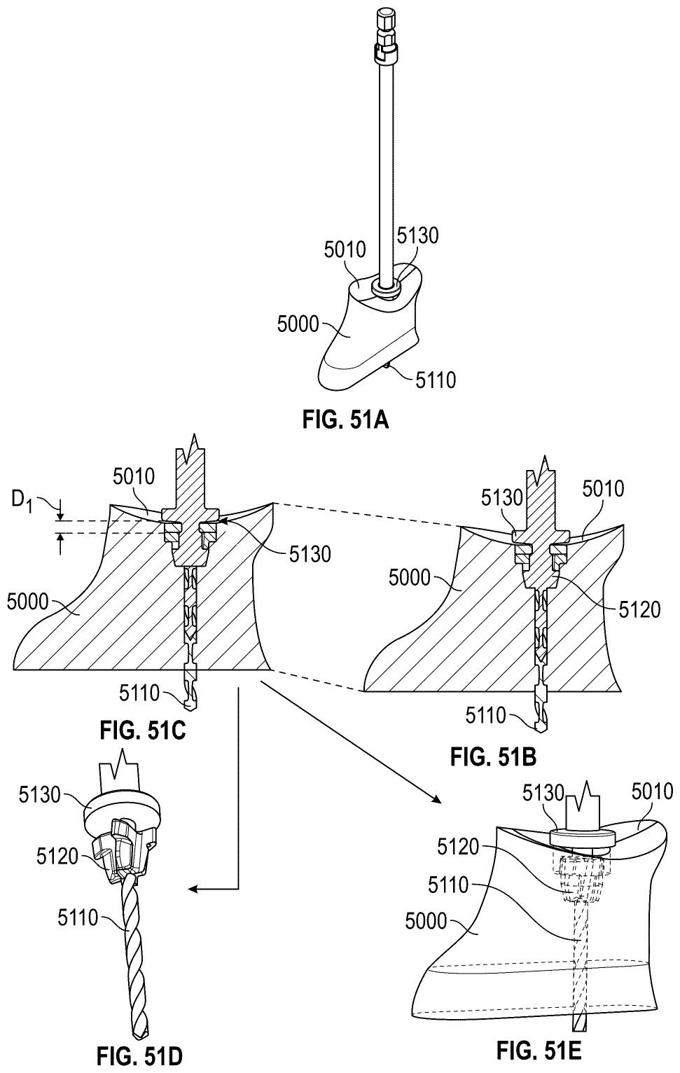

FIGS. 51A-51E illustrate different views or aspects related to a step of preparing glenoid bone 5000 for receiving a central anchor screw, for example 918 or as elsewhere described in this disclosure. Central anchor screws are described in connection with systems of at least FIGS. 9-20, 41A-41B, 46 and 48. Specifically, the practitioner uses a drill bit 5110 to drill a central hole into surface 5010 of bone 5000 for receiving central anchor screw 918. In some embodiments, bone 5000 is tapped for central anchor screw 918 having teeth diameters of approximately 6.5 mm, as previously described. In some embodiments, drill bit 5110 comprises a proximal counterboring portion 5120 having an increased radius compared to distal portions of drill bit 5110 to provide an increased diameter at the proximal end of the central hole to accommodate the profile of central anchor screw 918. In some embodiments, drill bit 5110 comprises a collar stop 5130 configured to prevent drill bit 5110 and/or counterboring portion 5120 from extending beyond a predetermined distance into surface 5010 of bone 5000 (e.g., to set the central anchor screw approximately 2.0 mm below glenoid surface 5010). However, the present disclosure is not so limited and adjustments (e.g., between −1.0 mm and +3.0 mm) may be made to this value.

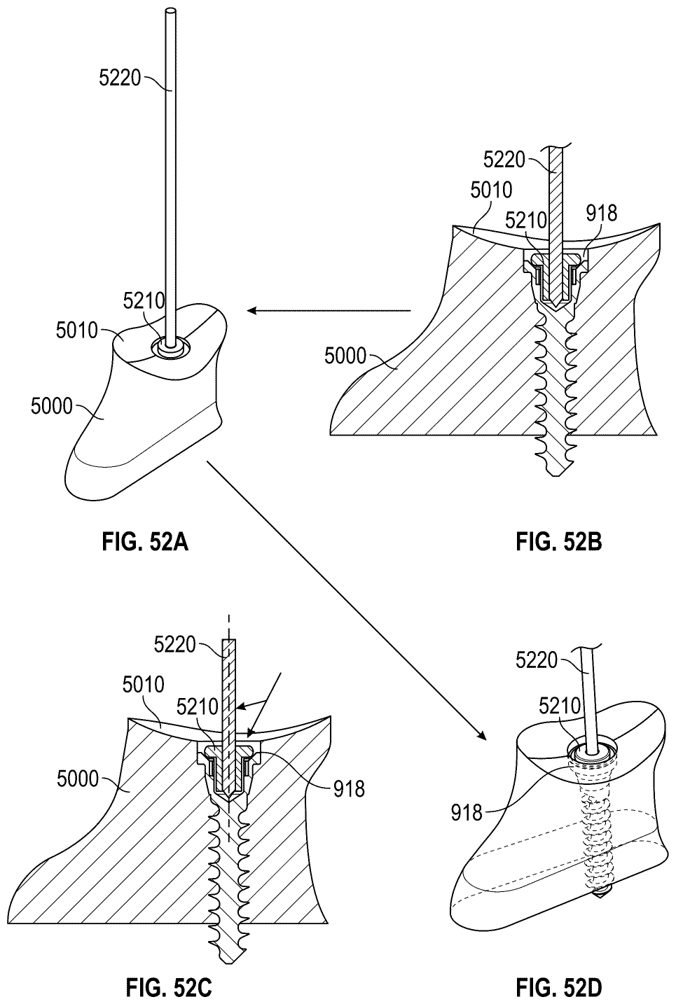

In FIGS. 52A-52D, central anchor screw 918 is properly disposed within the hole drilled by drill bit 5110 in FIGS. 51A-51E and a guide wire adapter 5210 is disposed or nestled into the minor diameter of the Torx opening in the head of central anchor screw 918. A guide wire 5220 is then coupled to guide wire adapter 5210. In some embodiments, guide wire 5220 has a diameter of 2.4 mm, though this disclosure is not so limited. In some embodiments, guide wire adapter 5210 and guide wire 5220 are a single, monolithic piece. In such embodiments, guide wire 5220 need not be coupled to guide wire adapter 5210 in a separate step. Guide wire 5220 may be utilized in a subsequent step, where glenoid face 5010 is reamed as required, e.g., for primary TSA, though the present disclosure is not so limited and preparation may be for any shoulder arthroplasty procedure.

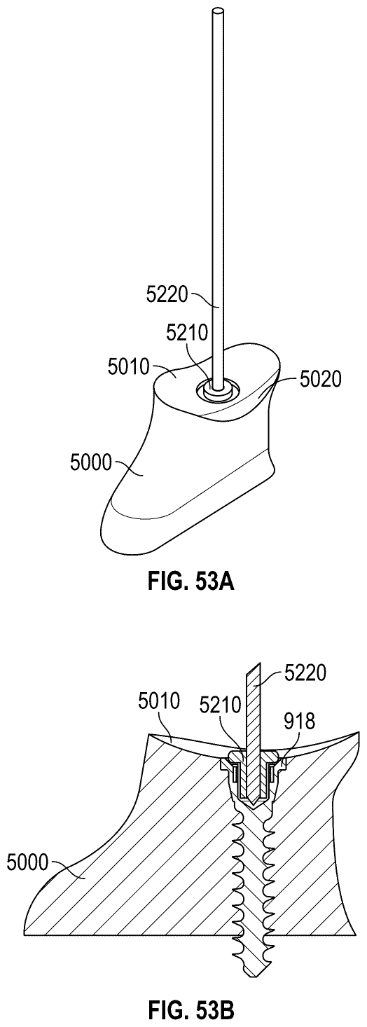

In FIGS. 53A and 53B, the practitioner uses a reamer (not shown), optionally threaded over guide wire 5220, to ream a portion of glenoid face 5010 and form a reamed surface 5020. Utilizing the same central anchor screw 918 to hold guide wire 5220 and/or adapter 5210 that will later anchor other components of the system ensures the surface 5020 is accurately and properly reamed for a tight and secure fit against bone 5000. Surface 5020 is for illustration only and any shape or sized reamed surface, in any orientation with respect to any other feature of bone 5000 is contemplated, as required in each procedure. In some embodiments, posterior wedge preparation may also be performed off guide wire (e.g., in applications utilizing AltiVate half wedges).

Once bone 5000 is properly prepared, guide wire 5220 and adapter 5210 may be removed and the remaining components of any compatible system described in this disclosure may be attached to central anchor screw 918 as previously described (see, e.g., at least FIGS. 9-20, 41A-41B, 46 and 48).

While FIG. 49 illustrates example procedures related to humeral bone and FIGS. 50A-53B illustrate examples related to scapular bone, the present disclosure also contemplates performing those example procedures on the reverse side(s) of the shoulder joint, for example on scapular and humeral bone, respectively.

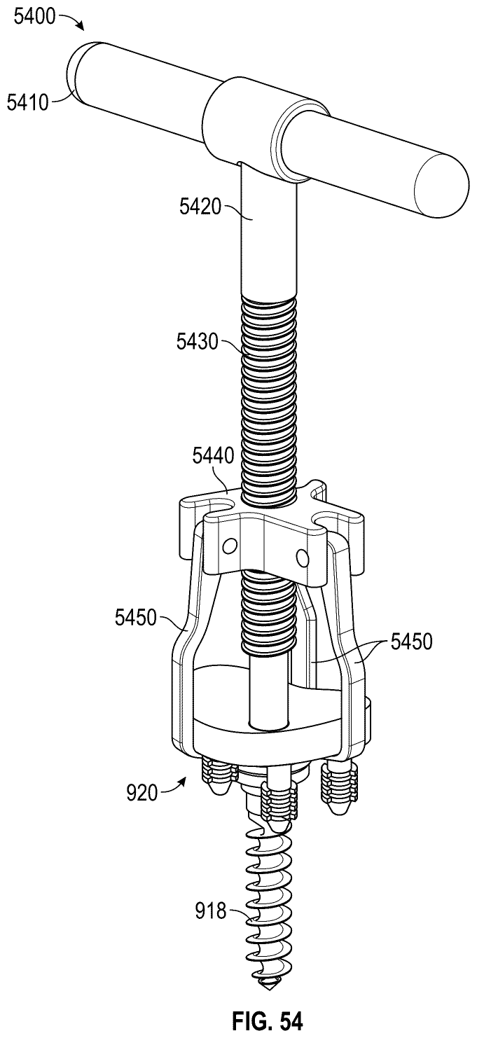

FIGS. 54 and 55A-55C illustrate different views of aspects related to implant removal, in accordance with some example embodiments. For example, in some systems according to at least FIGS. 9-20, 41A-41B, 46 and 48, central anchor screw 918 is ultimately coupled to an overlying baseplate, glenoid component or glenosphere component by coupling with the underside of metal disk-like component 924 disposed or fabricated therein (see, e.g., FIGS. 9-11). An extraction tool 5400 is specially designed to extract such overlying baseplate, glenoid component or glenosphere component as well as the metal disk-like component 924 disposed or fabricated therein in a single extraction.

Extraction tool 5400 comprises a handle 5410 and a rod portion 5420 extending from handle 5410, e.g., substantially forming a “T-shape.” At least a medial extent of rod portion 5420 is threaded and configured to engage with complementary threads in an aperture of a claw carrier 5440. Claw carrier 5440 is pivotally coupled to each of a plurality of claws 5450, each extending distal of claw carrier 5440 and configured to clamp under a respective portion of glenoid component 920 (e.g., shown for example only as the component having metal disk-like component 924 disposed in a bottom surface thereof). A distal portion 5430 of rod portion 5420 is threaded and configured to engage with complementary threads 1105 in aperture 1104 of metal disk-like component 924, which is machined or otherwise disposed in a bottom surface of glenoid component 920. Distal of distal portion 5430, rod portion 5420 tapers to a distal tip 5470. The decreased diameter of distal tap 5470 compared to distal threaded portion 5460 as well as to a minor diameter of the Torx opening in the head of central anchor screw 918 provides leverage at the interface between metal disk-like component 924 and central anchor screw 918, thereby allowing for an easy, clean extraction of glenoid component 920.

In some embodiments, a practitioner may hold handle portion 5410 and place at least distal tip 5470 of rod portion 5420 through aperture 929 in arcuate top surface 122. The practitioner may then rotate tool 5400 about rod portion 5420 by twisting on handle portion 5410 until distal threaded portion 5460 of rod portion 5420 threads through complementary threads 1105 in aperture 1104 of metal disk-like component 924 disposed in glenoid component 920.

### Methods of Use

The present disclosure also contemplates methods of using any component(s) described herein in any way described or intimated herein, for example in a surgical procedure, for example and not limitation, shoulder arthroplasty. Accordingly, while example features of several example methods of use are described below, the present disclosure is not so limited and contemplates methods including fewer, additional or alternative steps using any component(s) described herein in any way described or intimated herein. All methods described herein may be utilized in combination with, and/or may include, bone preparation processes as described in connection with any of at least FIGS. 49-53B.

For example, and not limitation, in some embodiments, a method may include anchoring an implant component to bone utilizing a center compression screw (see, e.g., FIGS. 1-8, 21-26B, 35A-35G, 42-45, 47 and 48) that, once properly implanted through a baseplate (see, e.g., FIGS. 1-4, 7, 8, 21-26B, 35A-35G, 42-45 and 48) or through an anchor boss (see, e.g., FIGS. 5, 6 and 47), imparts a compressive force to the baseplate or anchor boss, the baseplate or anchor boss, itself, being coupled and/or coupleable to the respective implant component.

In some other embodiments, a method may include anchoring an implant component to bone utilizing a central anchor screw (see, e.g., FIGS. 9-20) or a bossed central anchor screw (see, e.g., FIGS. 41A, 41B, 46 and 48) that is coupled and/or coupleable, from an underside of the baseplate, to the underside of the baseplate (see, e.g., FIGS. 12, 15-17, 41A, 41B and 48), the baseplate being coupled and/or coupleable to the implant component. In some other embodiments, the central anchor screw is directly coupled or coupleable, from an underside of the implant component, to the underside of the implant component itself (see, e.g., FIGS. 9-11, 13, 14, 18A-19B and 46).

In some embodiments, a metal disk-like component provides a key interface between the central anchor screw and the baseplate or implant component itself. Accordingly, a method may include coupling the central anchor screw to such a specially-designed metal disk-like component disposed and/or formed on or in the underside of the baseplate (see, e.g., FIGS. 12, 15-17 and 20) or of the implant component itself (see, e.g., FIGS. 9-11, 13, 14 and 18A-19B).

In some embodiments, a baseplate may not be utilized (see, e.g., FIGS. 5, 6, 9-11, 13, 14, 18A-19B, 46 and 47). In some such embodiments, a method may include securing an anchor boss into patient bone utilizing a compression screw, where the anchor boss is coupled to the implant component (see, e.g., FIGS. 5, 6 and 47). In some other such embodiments, such a method may include implanting a central anchor screw into patient bone and coupling the specially-designed metal disk-like component (see, e.g., FIGS. 18A-19B) or a central tapered portion having deflectable extensions disposed on or in the underside of the implant (see, e.g., FIGS. 46 and 47) to the central anchor screw.

In some other embodiments a baseplate may be utilized. In some such embodiments, a method may include disposing a central bossed portion extending from an underside of a baseplate into prepared patient bone (see, e.g., FIGS. 1-4, 7, 8, 21-35G, 44 and 45). In some other such embodiments, the baseplate does not comprise a bossed portion extending from its underside but, instead, comprises the specially-designed metal disk-like component disposed on or in its underside. In some such embodiments, such a method may include coupling the metal disk-like component to the central anchor screw (see, e.g., FIGS. 12, 15-17 and 20). In some other such embodiments, the baseplate comprises neither a central bossed portion extending from its underside nor the specially-designed metal disk-like component, instead, comprising an aperture having a bayonet-type locking feature (see, e.g., FIGS. 41A and 41B). In some such embodiments, a method may include coupling the specially-designed metal disk-like component to a bossed central anchor screw. In some other such embodiments, the baseplate comprises a central tapered portion extending from its topside (see, e.g., FIGS. 12, 20 and 48). In some such embodiments, a method may include taper or friction fitting this central tapered portion into a corresponding recess in a glenosphere component. In some embodiments, the baseplate comprises a wedged surface to address particularly-suited patient bone deficiencies and/or abnormalities (see, e.g., FIGS. 18A-20, 35A-35G, 37-41B and 48). In some such embodiments, a method may include disposing the wedged surface against the patient bone deficiencies and/or abnormalities.

In some embodiments, the baseplate may comprise one or more features to prevent rotation after implantation. In some such embodiments, a plurality of spikes extend from an underside of the baseplate (see, e.g., FIGS. 1 and 2). In some such embodiments, a method may include anchoring such spikes into bone peripheral to the compression screw. In some other such embodiments, a plurality of rough pegs extend from an underside of the baseplate (see, e.g., FIGS. 18A-19B, 46 and 47). In some such embodiments, a method may include anchoring the rough pegs into bone peripheral to the compression screw, central anchor screw or bossed central anchor screw. In some other such embodiments, the baseplate comprises a plurality of peripheral apertures configured to receive peripheral screws therethrough (see, e.g., FIGS. 3, 4, 7, 8, 12, 15-17, 20-35G, 41-45 and 48). In some such embodiments, a method may include anchoring the peripheral screws into bone peripheral to the compression screw, central anchor screw or bossed central anchor screw through the peripheral apertures.

In some embodiments, the implant component comprises a glenoid component (see, e.g., FIGS. 1-11, 13-16E, 18A-19B, 21-22B, 24-26B, 41A, 41B, 44, 46 and 47). In some such embodiments, the glenoid component comprises an upper plastic, e.g., poly, articulation portion and a lower metal portion (see, e.g., FIGS. 7, 8, 13 and 14). In some such embodiments, the glenoid component comprises a central tapered portion extending away from an underside thereof. In some such embodiments, a method may include taper or friction fitting such a central tapered portion within a recess and/or aperture disposed in a topside of a baseplate. In some embodiments where the glenoid component comprises plastic, e.g., poly, a metal tapered extension may extend from the central tapered portion. In some such embodiments, a method may include taper or friction fitting such a central tapered portion within the recess and/or aperture disposed in the topside of the baseplate (see, e.g., FIGS. 3-6). In some embodiments, the glenoid component comprises the specially-designed metal disk-like component disposed on or in its underside (see, e.g., FIGS. 9-11, 13, 14 and 18A-19B). In some such embodiments, a method may include interfacing the metal disk-like component directly with the central anchor screw. In some embodiments, the glenoid component comprises a central tapered portion having the deflectable extensions disposed on or in its underside (see, e.g., FIGS. 46 and 47). In some such embodiments, a method may include interfacing the central tapered portion and the deflectable extensions directly with the central anchor screw

In some embodiments, the implant component comprises a glenosphere component (see, e.g., FIGS. 12, 17, 20, 23A, 23B, 37-40B, 42, 43, 45 and 48), which may comprise a tapered recess. In some such embodiments, a method may include taper or friction fitting the tapered recess with any of: one tapered end of a dual-taper adapter (the other end of the dual-taper adapter being configured to taper or friction fit within an aperture in a top surface of the baseplate—see, e.g., FIG. 17—or within an aperture of a set screw disposed within the aperture in the top surface of the baseplate; a tapered end of a single-taper, threaded adapter (the other end of the single-taper comprising threads configured to engage with complementary threads in the aperture in the top surface of the baseplate—see, e.g., FIG. 45); or a central tapered portion extending away from a top surface of the baseplate (see, e.g., FIGS. 12, 20 and 48).

### Methods of Manufacture

The present disclosure also contemplates methods of manufacturing any component(s) described herein in any way described or intimated herein. Accordingly, while example features of several example methods of manufacture are described below, the present disclosure is not so limited and contemplates methods of manufacture including fewer, additional or alternative steps to form, provide, manufacture, fabricate and/or otherwise create any component(s) described herein.

This disclosure contemplates a variety of ways of accomplishing such manufacturing to provide the desired universality and interconvertibility of a surgical system, or kit, that allows securement of either of a glenoid or a glenosphere component thereto. General features common to various embodiments, or distinguishing various embodiments, will be described below. However, specific embodiments are also described in more detail in connection with the figures. This disclosure contemplates any method of manufacturing any component, or element thereof, described herein, for example and not limitation, providing, forming, fabricating, molding including but not limited to injecting molding or overmolding, extruding, stamping, deforming, casting, forging, milling, machining, printing including but not limited to 3-D printing any element and/or feature of any component, or the component itself to, thereby manufacture any component(s) described herein. Accordingly, manufacturing any component may comprise any one or more of these actions or steps, and contrarily, any one or more of these actions or steps may be considered manufacturing such a component and/or element thereof.

In some embodiments, a method of manufacture may comprise manufacturing an implant component and manufacturing a center compression screw, the implant component configured to be ultimately anchored to bone utilizing the center compression screw (see, e.g., FIGS. 1-8, 21-26B, 35A-35G, 42-45, 47 and 48). In some embodiments, such a center compression screw, once properly implanted through a baseplate (see, e.g., FIGS. 1-4, 7, 8, 21-26B, 35A-35G, 42-45 and 48) or through an anchor boss (see, e.g., FIGS. 5, 6 and 47), is configured to impart a compressive force to the baseplate or anchor boss, the baseplate or anchor boss, itself, being coupled and/or coupleable to the respective implant component.

In some other embodiments, a method of manufacture may comprise manufacturing an implant component and manufacturing a central anchor screw (see, e.g., FIGS. 9-20) and/or a bossed central anchor screw (see, e.g., FIGS. 41A, 41B, 46 and 48). In such embodiments, the implant component may be manufactured with a configuration allowing anchoring to bone utilizing the central anchor screw (see, e.g., FIGS. 9-20) or the bossed central anchor screw (see, e.g., FIGS. 41A, 41B, 46 and 48) that is coupled and/or coupleable, from an underside of the baseplate, to the underside of the baseplate (see, e.g., FIGS. 12, 15-17, 41A, 41B and 48). In some embodiments, the baseplate is manufactured with a configuration for coupling to the implant component. In some other embodiments, the central anchor screw is manufactured with a configuration for direct coupling, from an underside of the implant component, to the underside of the implant component itself (see, e.g., FIGS. 9-11, 13, 14, 18A-19B and 46).

In some embodiments, the central anchor screw may be manufactured with a configuration for coupling to a specially-designed metal disk-like component disposed and/or formed on or in the underside of the baseplate (see, e.g., FIGS. 12, 15-17 and 20) or of the implant component itself (see, e.g., FIGS. 9-11, 13, 14 and 18A-19B). The metal disk-like component is manufactured with a configuration that provides a key interface between the central anchor screw and the baseplate or implant component itself.

In some embodiments, a baseplate may not be utilized (see, e.g., FIGS. 5, 6, 9-11, 13, 14, 18A-19B, 46 and 47). In some such embodiments, a compression screw may be manufactured with a configuration to, instead, secure an anchor boss into patient bone. And the anchor boss may be manufactured with a configuration to couple to the implant component (see, e.g., FIGS. 5, 6 and 47). In some other such embodiments, a central anchor screw may be manufactured with a configuration for implantation into patient bone. And the specially-designed metal disk-like component (see, e.g., FIGS. 18A-19B) or a central tapered portion having deflectable extensions disposed on or in the underside of the implant (see, e.g., FIGS. 46 and 47) may be manufactured with respective configurations for coupling to the central anchor screw.

In some other embodiments, a baseplate may be utilized. In some such embodiments, the baseplate may be manufactured to include a central bossed portion extending from its underside and with a configuration to be disposed within prepared patient bone (see, e.g., FIGS. 1-4, 7, 8, 21-35G, 44 and 45). In some other such embodiments, the baseplate is not manufactured to include a bossed portion extending from its underside but is, instead, manufactured to include the specially-designed metal disk-like component disposed on or in its underside (see, e.g., FIGS. 12, 15-17 and 20). In some such embodiments, the metal disk-like component is manufactured with a configuration to couple to a central anchor screw. In some other such embodiments, the baseplate is manufactured to include neither a central bossed portion extending from its underside nor the specially-designed metal disk-like component, instead, being manufactured to include an aperture having a bayonet-type locking feature configured to couple a bossed central anchor screw (see, e.g., FIGS. 41A and 41B). In some other such embodiments, the baseplate is manufactured to include a central tapered portion extending from its topside (see, e.g., FIGS. 12, 20 and 48), which, in some cases, is manufactured with a configuration to taper or friction fit within a corresponding recess in a glenosphere component. Such methods may additionally include manufacturing such glenosphere components. In some embodiments, the baseplate is manufactured to include a wedged surface to address particularly-suited patient bone deficiencies and/or abnormalities (see, e.g., FIGS. 18A-20, 35A-35G, 37-41B and 48).

In some embodiments, the baseplate may be manufactured to include one or more features to prevent rotation after implantation. In some such embodiments, the baseplate is manufactured to include a plurality of spikes extend from an underside of the baseplate and anchor into bone peripheral to a compression screw (see, e.g., FIGS. 1 and 2). In some other such embodiments, the baseplate is manufactured to include a plurality of rough pegs extend from an underside of the baseplate and anchor into bone peripheral to a compression screw, central anchor screw or bossed central anchor screw (see, e.g., FIGS. 18A-19B, 46 and 47). In some other such embodiments, the baseplate is manufactured to include a plurality of peripheral apertures configured to receive peripheral screws therethrough that anchor into bone peripheral to the compression screw, central anchor screw or bossed central anchor screw (see, e.g., FIGS. 3, 4, 7, 8, 12, 15-17, 20-35G, 41-45 and 48).

In some embodiments, the implant component comprises a glenoid component (see, e.g., FIGS. 1-11, 13-16E, 18A-19B, 21-22B, 24-26B, 41A, 41B, 44, 46 and 47). In some embodiments, the glenoid component may comprise plastic, in some others metal. In some embodiments, a thickness of the glenoid component at its sulcus (i.e., the lowest point of the arcuate top surface and thinnest part of the glenoid component) may have an example thickness of 4.0-5.0 mm, for example approximately 4.16 mm. However, the disclosure is not so limited and any glenoid component described herein may also be manufactured to have any suitable thickness at its sulcus or at any other portion. In yet other embodiments, the glenoid component comprises an upper plastic, e.g., poly, articulation portion and a lower metal portion (see, e.g., FIGS. 7, 8, 13 and 14). In some such embodiments, the glenoid component is manufactured to include a central tapered portion extending away from an underside thereof that is configured to taper or friction fit within a recess and/or aperture disposed in a topside of the baseplate. In some embodiments where glenoid component comprises plastic, e.g., poly, the glenoid component may be manufactured to include a metal tapered extension extending from the central tapered portion and configured to taper or friction fit within the recess and/or aperture disposed in the topside of the baseplate (see, e.g., FIGS. 3-6). In some embodiments, the glenoid component is manufactured to include the specially-designed metal disk-like component disposed on or in its underside to provide a key interface directly with the central anchor screw (see, e.g., FIGS. 9-11, 13, 14 and 18A-19B). In some embodiments, the glenoid component is manufactured to include a central tapered portion having the deflectable extensions disposed on or in its underside to provide a key interface directly with the central anchor screw (see, e.g., FIGS. 46 and 47).

In some embodiments, the implant component comprises a glenosphere component (see, e.g., FIGS. 12, 17, 20, 23A, 23B, 37-40B, 42, 43, 45 and 48), which may be manufactured to include a tapered recess configured to taper or friction fit with any of: one tapered end of a dual-taper adapter (the other end of the dual-taper adapter being configured to taper or friction fit within an aperture in a top surface of the baseplate—see, e.g., FIG. 17—or within an aperture of a set screw disposed within the aperture in the top surface of the baseplate; a tapered end of a single-taper, threaded adapter (the other end of the single-taper comprising threads configured to engage with complementary threads in the aperture in the top surface of the baseplate—see, e.g., FIG. 45); or a central tapered portion extending away from a top surface of the baseplate (see, e.g., FIGS. 12, 20 and 48).

Such methods of manufacture may additionally include manufacturing any other element(s) of the system, including but not limited to such dual-taper adapters, set screws, single-taper threaded adapter, and/or central tapered portion extending away from the top surface of the baseplate.

The foregoing disclosure includes the best mode of the inventor for practicing the invention. It is apparent, however, that those skilled in the relevant art will recognize variations of the invention that are not described herein. While the invention is defined by the appended claims, the invention is not limited to the literal meaning of the claims, but also includes these variations.

1. A convertible shoulder arthroplasty system, comprising:
an implant component comprising at least one of:
a glenoid component comprising a concave outer surface, and/or
a glenosphere component comprising a convex outer surface;
a bone securement assembly comprising at least one of:
a first baseplate comprising a central bossed portion extending from an underside thereof and a central aperture disposed therethrough,
a second baseplate comprising a central bossed portion extending from a top side thereof and a central aperture disposed there through, and/or
an anchor boss comprising a central aperture extending therethrough; and
a central compression screw configured to be secured to a scapula through the central aperture and, thereby, securely compress the first baseplate, the second baseplate or the anchor boss against the scapula.

2. The system of claim 1, wherein the central bossed portion has one of a substantially tapered cylindrical shape and a substantially non-tapered cylindrical shape.

3. (canceled)

4. The system of claim 1, wherein at least one of the first baseplate and the second baseplate comprises a plurality of peripheral apertures, each configured to receive one of a plurality of peripheral bone screws.

5. (canceled)

6. The system of claim 1, wherein the first baseplate comprises at least one of:
one or more spikes extending from an underside thereof;
one or more smooth pegs extending from the underside; and/or
one or more rough pegs extending from the underside,
wherein the one or more spikes, the smooth pegs and/or the rough pegs are configured to be disposed into the patient bone and, thereby, prevent rotation of the glenoid baseplate with respect to the patient bone.

7. The system of claim 1, wherein:
the glenoid component comprises a central portion extending from an underside thereof configured to seat and friction-fit within the bossed portion of the first baseplate; and
the central portion comprises a plurality of vertically oriented grooves configured to increase friction between the central tapered portion and the bossed portion of the first baseplate.

8. (canceled)

9. The system of claim 1, wherein the central bossed portion of the first baseplate comprises a porous metal coating configured to aid in bone adhesion thereto and/or ingrowth therein.

10. The system of claim 1, wherein the glenoid component comprises;
a central portion extending from an underside thereof; and
a metal tapered extension coupled to a distal end of the central portion, the metal tapered extension comprising one or more substantially vertically oriented slots configured to allow the metal tapered extension to progress sufficiently into the bossed portion of the first baseplate to seat and friction-fit within the bossed portion of the first baseplate.

11. (canceled)

12. The system of claim 1, wherein an underside of the glenoid component comprises a recessed portion configured to receive at least a top portion of the first baseplate when the glenoid component is properly secured to the first baseplate.

13. The system of claim 1, wherein an underside of the glenoid component comprises:
one or more spikes extending therefrom;
one or more smooth pegs extending therefrom; and/or
one or more rough pegs extending therefrom,
wherein the one or more spikes, smooth pegs or rough pegs are configured to be disposed into the patient bone and, thereby, prevent rotation of the glenoid component with respect to the patient bone.

14. The system of claim 1, wherein the glenoid component comprises:
an upper portion comprising:
the concave surface, and
a patterned lower surface; and
a lower metal portion comprising:
a patterned upper surface configured to mate with the patterned lower surface of the upper portion, and
the underside of the glenoid component.

15. The system of claim 1, comprising a screw snap ring retaining the central compression screw within the bossed portion of the first baseplate in a pre-assembled state.

16. The system of claim 1, wherein the central compression screw is selected from a plurality of central compression screws each having a same head size but a different thread diameter compared to the other central compression screws such that the central bossed portion of the first baseplate is configured to accommodate each of the plurality of central compression screws.

17. The system of claim 1, wherein:
the central compression screw is selected from a plurality of central compression screws, each having a different thread diameter and a head size that varies with the thread diameter; and
the first baseplate is selected from a plurality of first baseplates, the respective central bossed portion of each first baseplate having a different size that accommodates the head size of one of the plurality of central compression screws.

18. The system of claim 1, wherein the first baseplate comprises a substantially planar top surface that is normal to an axial direction of extension of the central compression screw.

19. The system of claim 18, wherein an underside of the first baseplate extends in a plane that is rotated by a predetermined angle compared to the substantially planar top surface.

20. The system of claim 1, wherein the bossed portion of the first baseplate and/or of the second baseplate comprises threads configured to engage with complementary threads of a set screw configured to be secured within the central aperture over the central compression screw to prevent backout thereof.

21. The system of claim 20, wherein an inner portion of the set screw comprises a tapered recess configured to receive at least one of:
a central tapered portion extending from an underside of a glenoid component; and
a first tapered end of a dual-taper trunnion for the glenosphere component.

22. The system of claim 1, wherein the glenosphere component comprises a recess in an underside thereof configured to receive at least one of:
a first tapered end of a dual-taper trunnion for the glenosphere component, wherein a second tapered end of the dual-taper trunnion is configured to seat and friction fit within the bossed portion of the first baseplate;
a tapered end of a single-taper threaded trunnion for the glenosphere component, wherein a threaded end of the single-taper threaded trunnion is configured to thread into a complementary set of threads in the bossed portion of the first baseplate; and
the central bossed portion extending from the top side of the second baseplate.

23. The system of claim 1, wherein the glenosphere component comprises a substantially cylindrical skirt extending from the convex outer surface and configured to contact or substantially surround perimeter of the first baseplate or of the second baseplate and, thereby, share a load exerted on the first baseplate or on the second baseplate.

24. (canceled)

25. The system of claim 1, wherein the glenoid component comprises ultra-high molecular weight polyethylene (UHMWPE).

26. The system of claim 1, wherein at least one of the glenoid component, the glenosphere component, the first base plate and the second base plate comprises at least one of titanium (Ti) and cobalt.

27. (canceled)

28. (canceled)

29. (canceled)

30. A convertible shoulder arthroplasty system, comprising:
a central anchor screw comprising threads configured to bite into patient bone and provide a stand-alone anchor therein; and
one of:
a first glenoid component comprising a concave outer surface and an underside comprising a locking interface mating the glenoid component and the central anchor screw;
a first baseplate comprising a substantially planar top surface, a central aperture disposed therethrough, and an underside comprising a locking interface mating the first baseplate and the central anchor screw; and
a second baseplate comprising a central bossed portion extending from a top side thereof, a central aperture disposed therethrough, and an underside comprising a locking interface mating the second baseplate and the central anchor screw.

31. The system of claim 30, wherein the locking interface has a substantially circular form factor.

32. The system of claim 31, wherein the locking interface comprises:
a central aperture configured to receive an implant locking screw therethrough and a plurality of peripheral holes, each having a sidewall with a bottom-most portion that mechanically engages a head of the central anchor screw and decreases to a zero height toward the central aperture,
wherein distal threads of the implant locking screw are configured to mate with complementary threads in the head of the central anchor screw, thereby directly coupling the central anchor screw to the one of:
the underside of the first glenoid component,
the underside of the first baseplate, and
the underside of the second baseplate.

33. The system of claim 32, wherein the top surface of the glenoid baseplate comprises grooves and the underside of the second glenoid component comprises a plurality of ribs configured to snap and/or friction-fit within the grooves and, thereby secure the second glenoid component to the glenoid baseplate.

34. (canceled)

35. The system of claim 30, comprising the first baseplate, and further including a dual-taper trunnion and a glenosphere component comprising a recess in an underside thereof, wherein the recess is configured to receive a first tapered end of the dual-taper trunnion, wherein a second tapered end of the dual-taper trunnion is configured to seat and friction fit within the central aperture of the first baseplate.

36. The system of claim 30, wherein an underside of one of the first glenoid component, the first baseplate and the second baseplate extends in a plane that is rotated by a predetermined angle compared to a plane normal to an axial direction of extension of the central anchor screw.

37. (canceled)

38. (canceled)

39. (canceled)

40. (canceled)

41. (canceled)

42. A convertible shoulder arthroplasty system, comprising:
a glenoid component comprising a concave outer surface;
a baseplate comprising a central bossed portion extending from an underside thereof and a central aperture disposed therethrough; and
a central compression screw configured to be secured to a scapula through the central aperture and, thereby, securely compress the first baseplate against the scapula;
wherein the glenoid component comprises a central portion extending from an underside thereof configured to seat and friction-fit within the bossed portion of the first baseplate.

43. The system of claim 42, wherein the first baseplate comprises a plurality of peripheral apertures, each configured to receive one of a plurality of peripheral bone screws.

44. The system of claim 42, wherein the baseplate comprises at least one of:
one or more spikes extending from an underside thereof;
one or more smooth pegs extending from the underside; and/or
one or more rough pegs extending from the underside,
wherein the one or more spikes, the smooth pegs and/or the rough pegs are configured to be disposed into the patient bone and, thereby, prevent rotation of the baseplate with respect to the patient bone.

45. The system of claim 42, wherein the central portion extending from the underside of the glenoid component comprises a plurality of vertically oriented grooves configured to increase friction between the central portion and the central aperture of the first baseplate.

46. The system of claim 42, wherein the system comprises a retaining element configured to prevent backout of the central compression screw.

47. The system of claim 46, wherein the retaining element comprises one of:
a screw snap ring retaining the central compression screw within the central bossed portion in a pre-assembled state; and
a set screw comprising threads configured to engage with complementary threads of the bossed portion of the baseplate.

48. (canceled)

49. The system of claim 42, wherein an underside of the glenoid component comprises:
one or more spikes extending therefrom;
one or more smooth pegs extending therefrom; and/or
one or more rough pegs extending therefrom,
wherein the one or more spikes, smooth pegs or rough pegs are configured to be disposed into the patient bone and, thereby, prevent rotation of the glenoid component with respect to the patient bone.
